(19) United States
(12) Patent Application Publication
(10) Pub. No.: US 2008/0070924 A1
(43) Pub. Date: Mar. 20, 2008

(54) Novel Formulations For Opioid-Based Treatments Of Pain Comprising 1-(1,2-Disubstituted Piperidinyl)-4-Substituted Piperazine Derivatives

(76) Inventors: Frans Eduard Janssens, Rijmenam (BE); Francois Maria Sommen, Wortel (BE); Joseph Elisabeth Leenaerts, Rijkevorsel (BE); Yves Emiel Maria Van Roosbroeck, Hallaar (BE); Theo Frans Meert, Boom (BE)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/560,482

(22) PCT Filed: Jun. 7, 2004
(86) PCT No.: PCT/EP04/51050

§ 371 Date: Dec. 12, 2005

(30) Foreign Application Priority Data
Jun. 10, 2003 (EP) PCT/EP03/06118

(51) Int. Cl. A61K 31/496; A61P 25/04

(52) U.S. Cl. 514/253.09; 514/253.13

(57) ABSTRACT

This invention concerns novel formulations for opioid-based treatments of pain and/or nociception comprising opioid analgesics and 1-(1,2-disubstituted piperidinyl)-4-substituted piperazine derivatives having neurokinin antagonistic activity, in particular NK₁ antagonistic activity the use of said formulation for the manufacture of a medicament for the prevention and/or treatment of emesis, pain and/or nociception, in particular in acute and chronic pain treatments, more in particular in inflammatory, post-operative, emergency room (ER), breakthrough, neuropathic and cancer pain treatments and the use of an NK₁-receptor antagonist for the manufacture of a medicament for the prevention and/or treatment of respiratory depression and tolerance in opioid-based treatments of pain.

The pharmaceutical formulations according to the invention comprise NK₁-antagonists according to the general Formula (I)

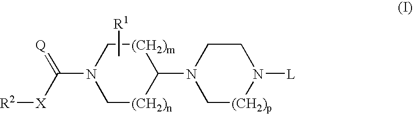

the pharmaceutically acceptable acid or base addition salts thereof, the stereochemically isomeric forms thereof, the N-oxide form thereof and prodrugs thereof, wherein all substituents are defined as in claim 1.

The pharmaceutical composition according to the invention reduces to a large extent a number of unwanted side-effects associated with opioid analgesics, in particular respiratory depression and tolerance, thereby increasing the total tolerability of said opioids in pain treatment.

### FIELD OF THE INVENTION

This invention concerns novel formulations for opioid-based treatments of pain and/or nociception comprising opioid analgesics and 1-(1,2-disubstituted piperidinyl)-4-substituted piperazine derivatives having neurokinin antagonistic activity, in particular NK₁ antagonistic activity, the use of said formulation for the manufacture of a medicament for the prevention and/or treatment of emesis, in particular nausea and vomiting, pain and/or nociception, in particular in opioid-based acute and chronic pain treatments, more in particular in inflammatory, post-operative, emergency room (ER), breakthrough, neuropathic and cancer pain treatments and the use of an NK₁-receptor antagonist for the manufacture of a medicament for the prevention and/or treatment of respiratory depression and tolerance in opioid-based treatments of pain.

### BACKGROUND OF THE INVENTION

Opioid analgesics are the cornerstone of pain treatment, especially in the segment of moderate to severe acute and chronic pain. However, side-effects such as nausea/vomiting, constipation, respiratory depression and tolerance limit their use. The lowering of the high incidence of nausea and vomiting with many clinically used opioids is particularly considered as a major unmet medical need.

Neurokinins belong to a family of short peptides that are widely distributed in the mammalian central and peripheral nervous system (Bertrand and Geppetti, Trends Pharmacol. Sci. 17:255-259 (1996); Lundberg, Can. J. Physiol. Pharmacol. 73:908-914 (1995); Maggi, Gen. Pharmacol 26:911-944 (1995); Regoli et al., Pharmacol. Rev. 46 (1994)). They share the common C-terminal sequence Phe-Xaa-Gly-Leu-Met-NH₂. Neurokinins released from peripheral sensory nerve endings are believed to be involved in neurogenic inflammation. In the spinal cord/central nervous system, neurokinins may play a role in pain transmission/perception and in some autonomic reflexes and behaviors. The three major neurokinins are Substance P (SP), Neurokinin A (NK_{A}) and Neurokinin B (NK_{B}) with preferential affinity for three distinct receptor subtypes, termed NK₁, NK₂, and NK₃, respectively. However, functional studies on cloned receptors suggest strong functional cross-interaction between the 3 neurokinins and their corresponding receptors (Maggi and Schwartz, Trends Pharmacol. Sci. 18: 351-355 (1997)). Species differences in structure of NK₁, receptors are responsible for species-related potency differences of NK₁, antagonists (Maggi, Gen. Pharmacol. 26:911-944 (1995); Regoli et al., Pharmacol. Rev. 46(4):551-599 (1994)). The human NK₁ receptor closely resembles the NK₁, receptor of guinea-pigs and gerbils but differs markedly from the NK₁, receptor of rodents. The development of neurokinin antagonists has led to date to a series of peptide compounds of which might be anticipated that they are metabolically too labile to be employed as pharmaceutically active substances (Longmore J. et al., DN&P 8(1):5-23 (1995)). NK₁-antagonists have been studied for a wide variety of indications including emesis, (stress-related) anxiety states, inflammatory responses, smooth muscle contraction and pain perception. NK₁-antagonists are in development for indications such as emesis, anxiety and depression, irritable bowel syndrome (IBS), circadian rhythm disturbances, visceral pain, neurogenic inflammation, asthma, micturition disorders, pancreatitis and nociception.

It has now surprisingly been found that a particular class of compounds with predominantly NK₁-activity reduces to a large extent a number of unwanted side-effects associated with opioid analgesics, thereby increasing the total tolerability of said opioids in pain treatment, in particular in opioid-based acute and chronic pain treatments, more in particular in inflammatory, post-operative, emergency room (ER), breakthrough, neuropathic and cancer pain treatments. More specifically, it was found in opioid-based treatments of pain that emesis was inhibited, respiratory depression was reduced, the tolerance for opioids was prevented and constipation was not worsened. Also, due to the intrinsic antinociceptive activity of NK₁-antagonists, even some increase in opioid efficacy is noted, thereby creating the option to reduce the opioid dose without effecting its analgesic action. Finally, by this combination, psychotropic properties were added to the analgesic efficacy by reducing stress, anxiety and depression.

### BACKGROUND PRIOR ART

1-(1,2-disubstituted piperidinyl)-4-substituted piperazine derivatives were disclosed in WO 97/16440-A1, published May 9, 1997 by Janssen Pharmaceutica N.V. for use as substance P antagonists.

Formulations containing NK₁-antagonists and opioid analgesics for the prevention and/or treatment of pain and/or nociception are disclosed in WO 96/20009 (Merck, Jul. 4, 1996) and WO 97/25988 (Eli Lilly, Jul. 24, 1997). There is no mentioning of the reduction of side-effects apart from emesis.

### DESCRIPTION OF THE INVENTION

The present invention relates to a pharmaceutical composition comprising a pharmaceutically acceptable carrier and, as active ingredients, a therapeutically effective amount of an opioid analgesic and a compound according to Formula (I)

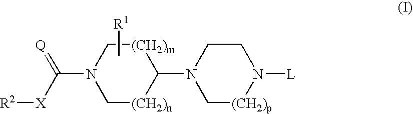

the pharmaceutically acceptable acid or base addition salts thereof, the stereochemically isomeric forms thereof, the N-oxide form thereof and the prodrugs thereof, wherein
n is 0, 1, or 2;
m is 1 or 2, provided that if m is 2, then n is 1;
p is 1 or 2;
═Q is ═O or ═NR³;
X is a covalent bond or a bivalent radical of formula —O—, —S—, —NR³—;
R¹ is Ar¹, Ar¹C₁₋₆alkyl or di(Ar¹)C₁₋₆alkyl, wherein each C₁₋₆alkyl group is optionally substituted with hydroxy, C₁₋₄alkyloxy, oxo or a ketalized oxo substituent of formula —O—CH₂—CH₂—O— or —O—CH₂ —CH₂—CH₂—O—;
R² is Ar², Ar²C₁₋₆alkyl, Het¹or Het¹C₁₋₆alkyl;
R³ is hydrogen or C₁₋₆alkyl;
L is hydrogen; Ar³; C₁₋₆alkyl; C₁₋₆alkyl substituted with 1 or 2 substituents selected from hydroxy, C₁₋₆alkyloxy, Ar³, Ar³C₁₋₆alkyloxy and Het²; C₃₋₆alkenyl; Ar³C₃₋₆alkenyl; di(Ar³)C₃₋₆alkenyl or a radical of formula

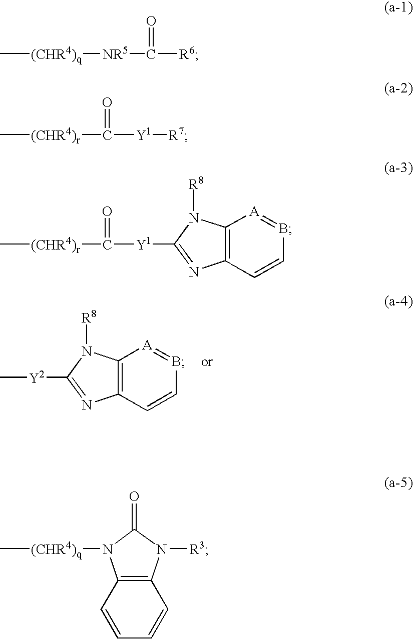

wherein each q independently is 2, 3 or 4;
each r is 0, 1, 2, 3 or 4;
each Y¹ independently is a covalent bond, —O— or NR³;
Y² is a covalent bond, C₁₋₄alkanediyl or -C₁₋₄alkyl NR³—;
each -A=B— independently is a bivalent radical of formula —CH═CH—, —N═CH— or —CH═N—;
each R⁴ independently is hydrogen, C₁₋₆alkyl, Ar² or Ar²C₁₋₆alkyl;
R⁵ is hydrogen, C₁₋₆alkyl or Ar³;
R⁶ is C₁₋₆alkyl, Ar³, Ar³C₁₋₆alkyl, di(Ar³)C₁₋₆alkyl, Ar³C₃₋₇cycloalkyl, or indolyl;
R⁷ is Ar³; Ar³C₁₋₆alkyl; di(Ar³)C₁₋₆alkyl; C₁₋₆alkyl; C₃₋₇cycloalkyl; C₃₋₇cycloalkyl substituted with Ar³; oxazolyl; oxazolyl substituted with halo or C₁₋₆alkyl; thiazolyl; thiazolyl substituted with halo or C₁₋₆alkyl; imidazolyl; imidazolyl substituted with Ar³, C₁₋₆alkyl, Ar³C₁₋₆alkyl or halo; indolinyl; indolinyl substituted with C₁₋₄alkyl; 2,3,4-trihydroquinolinyl; pyrrolidinyl or furanyl;
each R⁸ independently is hydrogen, C₁₋₆alkyl, C₃₋₇cycloalkyl or a radical of formula

-Alk-R¹¹ (b−1) or

-Alk-Z-R¹² (b−2);
wherein Alk is C₁₋₆ alkanediyl;
   Z is a bivalent radical of formula —O—, —S— or —NR³—;
   R¹¹ is phenyl; phenyl substituted with 1 or 2 substituents selected from halo, C₁₋₆alkyl or C₁₋₆alkyloxy; furanyl; furanyl substituted with 1 or 2 substituents selected from C₁₋₆alkyl or hydroxyC₁₋₆alkyl; thienyl; thienyl substituted with 1 or 2 substituents selected from halo or C₁₋₆alkyl; oxazolyl; oxazolyl substituted with 1 or 2 C₁₋₆alkyl substituents; thiazolyl; thiazolyl substituted with 1 or 2 C₁₋₆alkyl substituents; pyridinyl or pyridinyl substituted with 1 or 2 C₁₋₆alkyl substituents;
   R¹² is C₁₋₆alkyl or C₁₋₆alkyl substituted with hydroxy, carboxyl or C₁₋₆alkyloxycarbonyl;
Ar¹ is phenyl; phenyl substituted with 1, 2 or 3 substituents each independently selected from halo, C₁₋₄alkyl, haloC₁₋₄alkyl, cyano, aminocarbonyl, C₁₋₄alkyloxy or haloC₁₋₄alkyloxy;
Ar² is naphtalenyl; phenyl; phenyl substituted with 1, 2 or 3 substituents each independently selected from hydroxy, halo, cyano, nitro, amino, mono- or di(C₁₋₄alkyl)amino, C₁₋₄alkyl, haloC₁₋₄alkyl, C₁₋₄alkyloxy, haloC₁₋₄alkyloxy, carboxyl, C₁₋₄alkyloxycarbonyl, aminocarbonyl and mono- or di(C₁₋₄alkyl)aminocarbonyl;
Ar³ is phenyl or phenyl substituted with 1, 2 or 3 substituents selected from halo, hydroxy, amino, nitro, aminocarbonyl, C₁₋₆alkyl, haloC₁₋₆alkyl or C₁₋₆alkyloxy;
Het¹ is a monocyclic heterocycle selected from pyrrolyl, pyrazolyl, imidazolyl, furanyl, thienyl, oxazolyl, isoxazolyl, thiazolyl, isothiazolyl, pyridinyl, pyrimidinyl, pyrazinyl and pyridazinyl; or a bicyclic heterocycle selected from quinolinyl, quinoxalinyl, indolyl, benzimidazolyl, benzoxazolyl, benzisoxazolyl, benzothiazolyl, benzisothiazolyl, benzofuranyl and benzothienyl; each monocyclic and bicyclic heterocycle may optionally be substituted on a carbon atom by 1 or 2 substituents selected from halo, C₁₋₄alkyl or mono-, di- or tri(halo)methyl; and
Het² is a heterocycle selected from 1,4-dihydro-5-oxo-tetrazol-1-yl, imidazo[1,2-a]-pyridinyl, oxazolyl or imidazolyl; each of said heterocycles may be substituted with 1 or where possible 2 substituents selected from C₁₋₄alkyl and Ar³.

The heterocycles in the definition of Het¹ are preferably connected to the rest of the molecule, i.e. X, —C(═Q)— or C₁₋₆alkyl, by a carbon atom.

More in particular, the invention relates to a pharmaceutical composition comprising a pharmaceutically acceptable carrier and, as active ingredients, an opioid analgesic and a therapeutically effective amount of a compound according to Formula (I), the pharmaceutically acceptable acid or base addition salts thereof, the stereochemically isomeric forms thereof, the N-oxide form thereof and a prodrug thereof, wherein:

### L is hydrogen; C₁₋₆alkyl; C₁₋₆alkyl substituted with hydroxy; C₃₋₆alkenyl; Ar³;

Ar³C₁₋₆alkyl; di(Ar³)C₁₋₆alkyl; Ar³C₃₋₆alkenyl; di(Ar³)C₁₋₆alkenyl; or a radical of formula (a-1), (a-2), (a-4) or (a-5) wherein:
   R⁷ is Ar³; Ar³C₁₋₆alkyl; di(Ar³)C₁₋₆alkyl; C₁₋₆alkyl; C₃₋₇cycloalkyl; C₃₋₇cycloalkyl substituted with Ar³; oxazolyl; oxazolyl substituted with halo or C₁₋₆alkyl; thiazolyl; thiazolyl substituted with halo or C₁₋₆alkyl; imidazolyl; imidazolyl substituted with Ar³, C₁₋₆alkyl, Ar³C₁₋₆alkyl or halo; pyrrolidinyl or furanyl;
   Ar³ is is phenyl or phenyl substituted with 1, 2 or 3 substituents selected from halo, hydroxy, amino, aminocarbonyl, C₁₋₆alkyl, haloC₁₋₆alkyl or C₁₋₆alkyloxy;
   Het¹ is a monocyclic heterocycle selected from pyrrolyl, pyrazolyl, imidazolyl, furanyl, thienyl, oxazolyl, isoxazolyl, thiazolyl, isothiazolyl, pyridinyl, pyrimidinyl, pyrazinyl and pyridazinyl; or a bicyclic heterocycle selected from quinolinyl, benzimidazolyl, benzoxazolyl, benzisoxazolyl, benzothiazolyl, benzisothiazolyl, benzofuranyl and benzothienyl; each monocyclic and bicyclic heterocycle may optionally be substituted on a carbon atom by 1 or 2 substituents selected from halo, C₁₋₄alkyl or mono-, di- or tri(halo)methyl.

More in particular, the invention relates to a pharmaceutical composition comprising a pharmaceutically acceptable carrier and, as active ingredients, an opioid analgesic and a therapeutically effective amount of a compound according to Formula (I), the pharmaceutically acceptable acid or base addition salts thereof, the stereochemically isomeric forms thereof, the N-oxide form thereof and a prodrug thereof, wherein R¹ is Ar¹ methyl and attached to the 2-position or R¹ is Ar¹ and attached to the 3-position, as exemplified in either of the following formulas for compounds according to Formula (I) wherein m and n are equal to 1 and Ar is an unsubstituted phenyl. Preferably, Ar¹ methyl is an unsubstituted benzyl radical and Ar¹ is an unsubstituted phenyl radical.

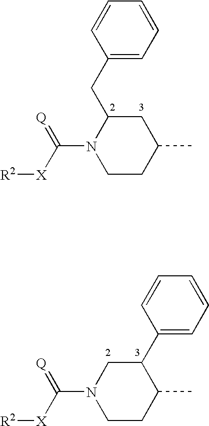

More in particular, the pharmaceutical composition comprises a compound according to the general Formula (I), the pharmaceutically acceptable acid or base addition salts thereof, the stereochemically isomeric forms thereof, the N-oxide form thereof and a prodrug thereof, wherein the R²-X—C(═Q)— moiety is 3,5-di-(trifluoromethyl)phenylcarbonyl.

More in particular, the pharmaceutical composition comprises a compound according to the general Formula (I), the pharmaceutically acceptable acid or base addition salts thereof, the stereochemically isomeric forms thereof, the N-oxide form thereof and a prodrug thereof, wherein R¹ is Ar¹C₁₋₆alkyl, R² is phenyl substituted with 2 substituents selected from methyl and trifluoromethyl, X is a covalent bond and ═Q is ═O.

More in particular, the pharmaceutical composition comprises a compound according to the general Formula (I), the pharmaceutically acceptable acid or base addition salts thereof, the stereochemically isomeric forms thereof, the N-oxide form thereof and a prodrug thereof, wherein n and m are 1 and p is 1 or 2.

More in particular, the pharmaceutical composition comprises a compound according to the general Formula (I), the pharmaceutically acceptable acid or base addition salts thereof, the stereochemically isomeric forms thereof, the N-oxide form thereof and a prodrug thereof, wherein R¹ is phenylmethyl; R² is phenyl substituted with 2 substituents selected from methyl or trifluoromethyl; n, m and p are 1; X is a covalent bond; and ═Q is ═O.

More in particular, the pharmaceutical composition comprises a compound according to the general Formula (I), the pharmaceutically acceptable acid or base addition salts thereof, the stereochemically isomeric forms thereof, the N-oxide form thereof and a prodrug thereof, wherein L is a radical of formula (a-2) wherein R⁴ is hydrogen or phenyl; r is 0 or 1; Y¹ is a covalent bond, —O— or —NH—; R⁷ is pyrrolidinyl; furanyl; 1-phenylcyclohexanyl; diphenylmethyl; or phenyl substituted with 1, 2 or 3 substituents each independently selected from methyl, methoxy or chloro.

Preferred compounds are those particular compounds that have a trans configuration.

More in particular, the pharmaceutical composition comprises a compound selected from the group of:
4-[1-[3,5-bis(trifluoromethyl)benzoyl]-2-(phenylmethyl)-4-piperidinyl]-N-(2,6-dimethylphenyl)-1-piperazine acetamide;
4-[1-[3,5-bis(trifluoromethyl)benzoyl]-2-(phenylmethyl)-4-piperidinyl]-N-(1-phenylcyclohexyl)-1-piperazine acetamide;
1-[3,5-bis(trifluoromethyl)benzoyl]-2-(phenylmethyl)-4-[4-[α-(1-pyrrolidinylcarbonyl)benzyl]-1-piperazinyl]piperidine;
1-[3,5-bis(trifluoromethyl)benzoyl]-4-[4-[1-[(2-methyl-5-oxazolyl)methyl]-1H-benzimidazol-2-yl]- 1-piperazinyl]-2-(phenylmethyl)piperidine;
4-[1-[3,5-bis(trifluoromethyl)benzoyl]-2-[(4-trifluoromethylphenyl)methyl]-4-piperidinyl]-N-(2,6-dimethylphenyl)-1-piperazine acetamide; and
4-[1-[3,5-bis(trifluoromethyl)benzoyl]-2-[(3,4-dichlorophenyl)methyl]-4-piperidinyl]-N-(2,6-dimethylphenyl)-1-piperazine acetamide.

Most in particular, the pharmaceutical composition comprises a compound selected from the group of:
(+)-(B)-trans-4-[1-[3,5-bis(trifluoromethyl)benzoyl]-2-(phenylmethyl)-4-piperidinyl]-N-(2,6-dimethylphenyl)-1-piperazine acetamide;
(−)-(B)-cis-4-[1-[3,5-bis(trifluoromethyl)benzoyl]-2-(phenylmethyl)-4-piperidinyl]-N-(2,6-dimethylphenyl)- 1-piperazine acetamide; and
-(+)-(B)-trans-4-[1-[3,5-bis(trifluoromethyl)benzoyl]-2-(phenylmethyl)-4-piperidinyl]-N-(2,6-dimethylphenyl)-1-piperazine acetamide, (L)-malic acid (1:1).

In the framework of this application, halo is generic to fluoro, chloro, bromo and iodo; C₂₋₄alkyl defines straight and branched chain saturated hydrocarbon radicals having from 2 to 4 carbon atoms such as, for example, ethyl, propyl, butyl, 1-methylethyl, 2-methylpropyl and the like; C₁₋₄alkyl is meant to include C₂₋₄alkyl and methyl; C₁₋₅alkyl is meant to include C₁₋₄alkyl and the higher homologues thereof having 5 carbon atoms such as, for example, pentyl, 2-methylbutyl and the like; C₁₋₆alkyl is meant to include C₁₋₅alkyl and the higher homologues thereof having 6 carbon atoms such as, for example, hexyl, 2-methylpentyl and the like; C₁₋₄alkanediyl defines bivalent straight and branched chain saturated hydrocarbon radicals having from 1 to 4 carbon atoms such as, for example, methylene, 1,2-ethanediyl, 1,3-propanediyl, 1,4-butanediyl, and the like; C₁₋₆alkanediyl is meant to include C₁₋₄alkanediyl and the higher homologues thereof having form 5 to 6 carbon atoms such as, for example, 1,5-pentanediyl, 1,6-hexanediyl and the like; C₃₋₆alkenyl defines straight and branched chain hydrocarbon radicals containing one double bond and having from 3 to 6 carbon atoms such as, for example, 2-propenyl, 3-butenyl, 2-butenyl, 2-pentenyl, 3-pentenyl, 3-methyl-2-butenyl, 3-hexenyl and the like; and the carbon of said C₃₋₆alkenyl connected to the nitrogen atom of the piperazine or homopiperazine preferably is saturated.

As used in the foregoing definitions and hereinafter, haloC₁₋₄alkyl is defined as mono- or polyhalosubstituted C₁₋₄alkyl, in particular C₁₋₄alkyl substituted with 1 to 6 halogen atoms, more in particular difluoro- or trifluoromethyl.

The pharmaceutically acceptable salts are defined to comprise the therapeutically active non-toxic acid addition salts forms that the compounds according to Formula (I) are able to form. Said salts can be obtained by treating the base form of the compounds according to Formula (I) with appropriate acids, for example inorganic acids, for example hydrohalic acid, in particular hydrochloric acid, hydrobromic acid, sulfuric acid, nitric acid and phosphoric acid ; organic acids, for example acetic acid, hydroxyacetic acid, propanoic acid, lactic acid, pyruvic acid, oxalic acid, malonic acid, succinic acid, maleic acid, fumaric acid, malic acid, tartaric acid, citric acid, methanesulfonic acid, ethanesulfonic acid, benzenesulfonic acid, p-toluenesulfonic acid, cyclamic acid, salicylic acid, p-aminosalicylic acid and pamoic acid.

The compounds according to Formula (I) containing acidic protons may also be converted into their therapeutically active non-toxic metal or amine addition salts forms by treatment with appropriate organic and inorganic bases. Appropriate base salts forms comprise, for example, the ammonium salts, the alkaline and earth alkaline metal salts, in particular lithium, sodium, potassium, magnesium and calcium salts, salts with organic bases, e.g. the benzathine, N-methyl-D-glucamine, hybramine salts, and salts with amino acids, for example arginine and lysine.

Conversely, said salt forms can be converted into the free forms by treatment with an appropriate base or acid.

The term addition salt as used in the framework of this application also comprises the solvates that the compounds according to Formula (I) as well as the salts thereof, are able to form. Such solvates are, for example, hydrates and alcoholates.

The N-oxide forms of the compounds according to Formula (I) are meant to comprise those compounds according to Formula (I) wherein one or several nitrogen atoms are oxidized to the so-called N-oxide, particularly those N-oxides wherein one or more tertiary nitrogens (e.g of the piperazinyl or piperidinyl radical) are N-oxidized. Such N-oxides can easily be obtained by a skilled person without any inventive skills and they are obvious alternatives for the compounds according to Formula (I) since these compounds are metabolites, which are formed by oxidation in the human body upon uptake. As is generally known, oxidation is normally the first step involved in drug metabolism (Textbook of Organic Medicinal and Pharmaceutical Chemistry, 1977, pages 70-75). As is also generally known, the metabolite form of a compound can also be administered to a human instead of the compound per se, with much the same effects.

The compounds according to Formula (I) possess at least 2 oxydizable nitrogens (tertiary amines moieties). It is therefore highly likely that N-oxides are to form in the human metabolism.

The compounds according to Formula (I) may be converted to the corresponding N-oxide forms following art-known procedures for converting a trivalent nitrogen into its N-oxide form. Said N-oxidation reaction may generally be carried out by reacting the starting material according to Formula (I) with an appropriate organic or inorganic peroxide. Appropriate inorganic peroxides comprise, for example, hydrogen peroxide, alkali metal or earth alkaline metal peroxides, e.g. sodium peroxide, potassium peroxide; appropriate organic peroxides may comprise peroxy acids such as, for example, benzenecarboperoxoic acid or halo substituted benzenecarboperoxoic acid, e.g. 3-chlorobenzenecarboperoxoic acid, peroxoalkanoic acids, e.g. peroxoacetic acid, alkylhydroperoxides, e.g. tert-butyl hydroperoxide. Suitable solvents are, for example, water, lower alkanols, e.g. ethanol and the like, hydrocarbons, e.g. toluene, ketones, e.g. 2-butanone, halogenated hydrocarbons, e.g. dichloromethane, and mixtures of such solvents.

The term “stereochemically isomeric forms” as used hereinbefore defines all the possible isomeric forms that the compounds according to Formula (I) may possess. Unless otherwise mentioned or indicated, the chemical designation of compounds denotes the mixture of all possible stereochemically isomeric forms, said mixtures containing all diastereomers and enantiomers of the basic molecular structure. More in particular, stereogenic centers may have the R- or S-configuration; substituents on bivalent cyclic (partially) saturated radicals may have either the cis- or trans-configuration. Compounds encompassing double bonds can have an E or Z-stereochemistry at said double bond. Stereochemically isomeric forms of the compounds according to Formula (I) are obviously intended to be embraced within the scope of this invention.

Following CAS nomenclature conventions, when two stereogenic centers of known absolute configuration are present in a molecule, an R or S descriptor is assigned (based on Cahn-Ingold-Prelog sequence rule) to the lowest-numbered chiral center, the reference center. The configuration of the second stereogenic center is indicated using relative descriptors [R*,R*] or [R*,S*], where R* is always specified as the reference center and [R*,R*] indicates centers with the same chirality and [R*,S*] indicates centers of unlike chirality. For example, if the lowest-numbered chiral center in the molecule has an S configuration and the second center is R, the stereo descriptor would be specified as S-[R*,S*]. If “α” and “β” are used : the position of the highest priority substituent on the asymmetric carbon atom in the ring system having the lowest ring number, is arbitrarily always in the “α” position of the mean plane determined by the ring system. The position of the highest priority substituent on the other asymmetric carbon atom in the ring system (hydrogen atom in compounds according to Formula (I)) relative to the position of the highest priority substituent on the reference atom is denominated “α”, if it is on the same side of the mean plane determined by the ring system, or “β”, if it is on the other side of the mean plane determined by the ring system.

Compounds according to Formula (I) and some of the intermediate compounds have at least two stereogenic centers in their structure.

The invention also comprises pharmaceutical compositions according to the invention comprising derivative compounds (usually called “pro-drugs”) of the pharmacologically-active compounds according to the invention, which are degraded in vivo to yield the compounds according to the invention. Pro-drugs are usually (but not always) of lower potency at the target receptor than the compounds to which they are degraded. Pro-drugs are particularly useful when the desired compound has chemical or physical properties that make its administration difficult or inefficient. For example, the desired compound may be only poorly soluble, it may be poorly transported across the mucosal epithelium, or it may have an undesirably short plasma half-life. Further discussion on pro-drugs may be found in Stella, V. J. et al., “Prodrugs”, Drug Delivery Systems, 1985, pp. 112-176, and Drugs, 1985, 29, pp. 455-473.

Pro-drugs forms of the pharmacologically-active compounds according to the invention will generally be compounds according to Formula (I), the pharmaceutically acceptable acid or base addition salts thereof, the stereochemically isomeric forms thereof and the N-oxide form thereof, having an acid group which is esterified or amidated. Included in such esterified acid groups are groups of the formula —COOR^{x}, where R^{x} is a C₁₋₆alkyl, phenyl, benzyl or one of the following groups:

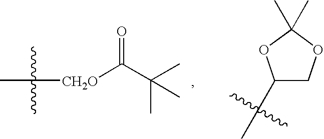

Amidated groups include groups of the formula —CONR^{y}R^{z}, wherein R^{y} is H, C₁₋₆alkyl, phenyl or benzyl and R^{z} is —OH, H, C₁₋₆alkyl, phenyl or benzyl. Compounds according to the invention having an amino group may be derivatised with a ketone or an aldehyde such as formaldehyde to form a Mannich base. This base will hydrolyze with first order kinetics in aqueous solution.

The compounds according to Formula (I) as prepared in the processes described below may be synthesized in the form of racemic mixtures of enantiomers that can be separated from one another following art-known resolution procedures. The racemic compounds according to Formula (I) may be converted into the corresponding diastereomeric salt forms by reaction with a suitable chiral acid. Said diastereomeric salt forms are subsequently separated, for example, by selective or fractional crystallization and the enantiomers are liberated therefrom by alkali. An alternative manner of separating the enantiomeric forms of the compounds according to Formula (I) involves liquid chromatography using a chiral stationary phase. Said pure stereochemically isomeric forms may also be derived from the corresponding pure stereochemically isomeric forms of the appropriate starting materials, provided that the reaction occurs stereospecifically. Preferably if a specific stereoisomer is desired, said compound would be synthesized by stereospecific methods of preparation. These methods will advantageously employ enantiomerically pure starting materials.

In the framework of this application, the term opioid means opium-like or morphine-like in terms of pharmacological action. The broad group of opium alkaloids, synthetic derivatives related to the opium alkaloids, and the many naturally occuring and synthetic peptides with morphine-like pharmacological effects is called opioids. In addition to having pharmacological effects similar to those of morphine, a compound must be antagonized by an opioid antagonist such as naloxone to be classified as an opioid. The neuronally located proteins to which opioid agents bind to initiate a biological response are called opioid receptors. Opioids can act peripherally and centrally.

Suitable opioids or opioid analgesics for use in the present invention include one or more compounds selected from the group of alfentanil, buprenorphine, butorphanol, carfentanil, codeine, diacetylmorphine, dihydrocodeine, fentanyl, hydrocodone, hydromorphone, levorphanol, lofentanil, meperidine, methadone, morphine, nalbuphine, oxycodone, oxymorphone, pentazocine, propoxyphene, remifentanil and sufentanil; and pharmaceutical acceptable salts and derivatives thereof.

Because of their widespread use as analgesics, preferred opioid analgesics of use in the present invention are one or more compounds selected from the group of oxycodone, codeine, morphine, fentanyl, buprenorphine, hydrocodone, hydromorphone and pharmaceutical acceptable salts and derivatives thereof..

Suitable pharmaceutically acceptable salts of the opioid analgesics of use in the present invention include those salts described above in relation to the salts of the NK₁-antagonist.

Preferred salts of opioid analgesics of use in the present invention include alfentanil hydrochloride, buprenorphine hydrochloride, butorphanol tartrate, codeine phosphate, codeine sulphate, diacetylmorphine hydrochloride, dihydrocodeine bitartrate, fentanyl citrate, hydrocodone bitartrate, hydromorphone hydrochloride, levorphanol tartrate, meperidine hydrochloride, methadone hydrochloride, morphine sulphate, morphine hydochloride, morphine tartrate, nalbuphine hydrochloride, oxymorphone hydrochloride, pentazocine hydrochloride, propoxyphene hydrochloride and propoxyphene napsylate (2-naphthalene sulphonic acid (1:1) monohydrate).

More particular preferred opioid analgesics of use in the present invention are morphine sulphate and fentanyl citrate.

### Pharmacology

The compounds according to Formula (I) are potent inhibitors of neurokinin-mediated effects, in particular those mediated via the NK₁ receptor, and may therefore be described as neurokinin antagonists, especially as substance P antagonists, as indicated in vitro by the antagonism of substance P-induced relaxation of pig coronary arteries which is described hereinafter. The binding affinity of the present compounds for the human, guinea-pig and gerbil neurokinin receptors may be determined in vitro in a receptor binding test using ³H-substance-P as radioligand. The subject compounds also show substance-P antagonistic activity in vivo as may be evidenced by, for instance, the antagonism of substance P-induced plasma extravasation in guinea-pigs, or the antagonism of drug-induced emesis in ferrets (Watson et al., Br. J Pharmacol. 115:84-94 (1995)).

The combination of an opioid analgesic with an NK₁, antagonist results in improved efficacy. Additional to the gain in efficacy, this combination also reduces several of the side-effects currently present with clinically used opioids. NK₁ receptor antagonists potentiating the analgesic activity of opioids require lower doses, resulting in a reduced risk of opioid side-effects, in particular emesis, respiratory depression and tolerance. But additionally it's seen that at similar doses (not lower opioid doses) there are also benefits of adding NK1 to opioid.

Respiratory depression is the most serious side effect of opioid analgesics and is the primal cause of death from overdose. Opioids decrease the sensitivity of chemoreceptors in the brainstem to carbon dioxide, a normal stimulus of ventilatory reflexes. The result is a blunting of the ventilatory response to increases in the carbon dioxide tension (P_{CO}_{₂}) in blood and cerebrospinal fluid. At equally effective analgesic doses, most opioids produce a similar degree of respiratory depression, as shown by an elevation in the blood P_{CO}_{₂}. This effect is at least additive to that produced by other drugs that depress CNS functions, including general anesthetics and sedative-hypnotics. The mild respiratory depression produced by therapeutic doses of opioids is normally of little consequence. However, opioid analgesics must be used cautiously in patients with traumatic head injuries, with emphysema and who are morbidly obese. At three to five times its usual analgesic dose, morphine can cause respiratory arrest in the nontolerant patient. In contrast, much higher doses will have minimal respiratory effects in morphine-tolerant individuals.

Tolerance refers to a reduced drug effect with repeated use and/or a need for higher doses to produce the same effect. Because tolerance does not occur to the same extent for all effects, drug abusers who take increasing amounts of drugs risk exposure to those effects to which tolerance does not develop. Tolerance develops to many of the effects of opioids. With repeated drug administration, larger doses are necessary to produce the same pharmacological response. The rate of tolerance development varies with the affected tissue of organ. Tolerance develops rapidly to the antiemetic effects of opioids; more gradually to their analgesic, endocrine and respiratory depressant effects; and virtually not at all to their constipating and miotic effects.

The compounds according to the invention have shown to reduce unwanted side-effects induced by opioids. Such reduction can be tested by in vivo testing using several species (e.g. ferrets, gerbils, rats, guinea pigs) and several pain models, covering pain models aiming at different states of acute and chronic pain, as well as animal models aiming to profile opioid side effects (such as opioid-induced emesis, GI transit and respiratory depression). For instance, the compounds of the present invention:
were able to inhibit the opioid-induced emesis in several species;
did not reduce the antinociceptive properties of opioids in models of acute, visceral and high intensity pain;
had an additive effect on the antinociceptive properties of opioids in models of inflammatory and chronic neuropathic pain;
reduced the respiratory depression induced by opioids in several species;
were able to reduce and overcome the tolerance observed with opioids daily administered in a model of chronic neuropathic pain;
did not interfere with the discriminative central narcotic effects of opioids;
had no effect on the pharmacokinetics of opioids when administered concomitantly.
This excludes pharmacokinetic interactions as the origin of the pharmacological effects observed.

The present invention therefore also relates to the use of a pharmaceutical composition according to the invention for the manufacture of a medicament for the prevention and/or treatment of pain and/or nociception.

In particular, the present invention relates to the use of a pharmaceutical composition according to the invention for the manufacture of a medicament for the opioid-based prevention and/or treatment of acute and chronic pain, more in particular in inflammatory, post-operative, emergency room (ER), breakthrough, neuropathic and cancer pain treatments.

The present invention further relates to the use of a pharmaceutical composition according to the invention for the manufacture of a medicament for the prevention and/or treatment of emesis in opioid-based treatments of pain.

The present invention further relates to the use of a pharmaceutical composition according to the invention for the manufacture of a medicament for the prevention and/or treatment of nausea and vomiting in opioid-based treatments of pain.

The present invention also relates to the use of an NK₁-receptor antagonist, in particular an NK₁-receptor antagonist according to Formula (I), the pharmaceutically acceptable acid or base addition salts thereof, the stereochemically isomeric forms thereof, the N-oxide form thereof and prodrugs thereof, for the manufacture of a medicament for the prevention and/or treatment of respiratory depression in opioid-based treatments of pain.

The present invention also relates to the use of an NK₁-receptor antagonist, in particular an NK₁-receptor antagonist according to Formula (I), the pharmaceutically acceptable acid or base addition salts thereof, the stereochemically isomeric forms thereof, the N-oxide form thereof and prodrugs thereof, for the manufacture of a medicament for reducing and/or overcoming the tolerance observed with opioids, e.g. when daily administered in chronic neuropathic pain.

To prepare the pharmaceutical compositions of this invention, an effective amount of the active ingredient, optionally in addition salt form, is combined in intimate admixture with a pharmaceutically acceptable carrier, which carrier may take a wide variety of forms depending on the form of preparation desired for administration. The pharmaceutical compositions are desirable in unitary dosage form suitable, in particular, for administration orally, rectally, percutaneously, by parenteral injection or by inhalation. For example, in preparing the compositions in oral dosage form, any of the usual pharmaceutical media may be employed such as, for example, water, glycols, oils, alcohols and the like in the case of oral liquid preparations such as suspensions, syrups, elixirs, emulsions and solutions; or solid carriers such as starches, sugars, kaolin, diluents, lubricants, binders, disintegrating agents and the like in the case of powders, pills, capsules and tablets. Because of their ease in administration, tablets and capsules represent the most advantageous oral dosage unit forms in which case solid pharmaceutical carriers are obviously employed. For parenteral compositions, the carrier will usually comprise sterile water, at least in large part, though other ingredients, for example, to aid solubility, may be included. Injectable solutions, for example, may be prepared in which the carrier comprises saline solution, glucose solution or a mixture of saline and glucose solution. Injectable suspensions may also be prepared in which case appropriate liquid carriers, suspending agents and the like may be employed. Also included are solid form preparations that are intended to be converted, shortly before use, to liquid form preparations. In the compositions suitable for percutaneous administration, the carrier optionally comprises a penetration enhancing agent and/or a suitable wetting agent, optionally combined with suitable additives of any nature in minor proportions, which additives do not introduce a significant deleterious effect on the skin. Said additives may facilitate the administration to the skin and/or may be helpful for preparing the desired compositions. These compositions may be administered in various ways, e.g., as a transdermal patch, as a spot-on, as an ointment. Other compositions may be compositions in a form suitable for sublingual, intranasal or pulmonary application or suitable as eye droplets.

It is especially advantageous to formulate the aforementioned pharmaceutical compositions in unit dosage form for ease of administration and uniformity of dosage. Unit dosage form as used herein refers to physically discrete units suitable as unitary dosages, each unit containing a predetermined quantity of active ingredient calculated to produce the desired therapeutic effect in association with the required pharmaceutical carrier. Examples of such unit dosage forms are tablets (including scored or coated tablets), capsules, pills, powder packets, wafers, suppositories, injectable solutions or suspensions and the like, and segregated multiples thereof.

Since the compounds according to the invention are potent orally administrable NK₁, antagonists, pharmaceutical compositions comprising said compounds for administration orally are especially advantageous.

The NK₁-receptor antagonist and the opioid analgesic may be formulated in a single pharmaceutical composition or alternatively in individual pharmaceutical compositions for simultaneous, separate or sequential use in accordance with the present invention. The pharmaceutical composition may also be a product comprising the NK₁-receptor antagonist and the opioid analgesic as separate unit dosages.

When administered in combination, either as a single or as separate pharmaceutical composition(s), the NK₁-receptor antagonist and the opioid analgesic are presented in a ratio which is consistent with the manifestation of the desired effect. In particular, the ratio by weight of the NK₁-antagonist to the opioid analgesic will suitably be approximately 1 to 1. Preferably, this ratio will be between 0.001 to 1 and 1000 to 1, and especially between 0.01 to 1 and 100 to 1.

A suitable dosage level for the NK₁-receptor antagonist is about 0.001 to 25 mg/kg per day, preferably about 0.005 to 10 mg/kg per day, and especially about 0.005 to 5 mg/kg day. The compounds may be administered on a regimen of up to 6 times per day, preferably 1 to 4 times per day.

The opioid analgesic may be administered at a dosage level up to conventional dosage levels for such analgesics, but preferably at a reduced level in accordance with the present invention. Suitable dosage levels will depend upon the analgesic effect of the chosen opioid analgesic, but typically suitable levels will be about 0.001 to 25 mg/kg per day, preferably 0.005 to 10 mg/kg per day, and especially 0.005 to 5 mg/kg day. The compound may be administered on a regimen of up to 6 times per day, preferably 1 to 4 times per day.

It will be appreciated that the amount of an NK₁-receptor antagonist and an opioid analgesic required for use in the prevention and/or treatment of pain and nociception will vary not only with the particular compounds or compositions selected but also with the route of administration, the nature of the condition being treated, and the age and condition of the human in need of such a treatment, and will ultimately be at the discretion of the attendant physician.

### Chemistry

The compounds according to the invention can generally be prepared by a succession of steps, each of which is known to the skilled person.

The preparation of these compounds and their intermediates is also described in WO 97/16440-A1, published May 9, 1997 by Janssen Pharmaceutica N.V, which is disclosed herein by reference as well as in other publications mentioned in WO 97/16440-A1, such as, e.g. EP-0,532,456-A.

The following examples are intended to illustrate and not to limit the scope of the present invention.

### Experimental Section

### Hereinafter “RT” means room temperature, “THF” means tetrahydrofuran, “DIPE” means diisopropylether, “DCM” means dichloromethane and “DMF” means N,N-dimethylformamide.

### A. Preparation of the Intermediate Compounds

### EXAMPLE A.1

a) A mixture of (±)-1,1-dimethyl 7-(phenylmethyl)-1,4-dioxa-8-azaspiro[4,5]decane-8-carboxylate (13 g; prepared according to the method described in EP-A-532,456) in HCl (6 N; 130 ml) was stirred and refluxed for 3 hours. The reaction mixture was cooled, alkalized with aqueous NaOH (50%) and extracted with DCM. The organic layer was separated, dried, filtered, and the filtrate, which contained (±)-2-(phenyl-methyl)-4-piperidinone (intermediate 1), was used in next reaction step.
b) A mixture of the filtrate obtained in the previous reaction step, 3,5-dimethylbenzoyl chloride (7.4 g) and triethylamine (11 ml) was stirred overnight at RT. The reaction mixture was extracted with dilute NaOH solution. The organic layer was separated, dried, filtered and the solvent evaporated. The residue was crystallized from DIPE. The precipitate was filtered off and dried, yielding 7.44 g (58%) of (±)-1-(3,5-dimethyl-benzoyl)-2-(phenylmethyl)-4-piperidinone (intermediate 2; mp. 107.8° C.).

### EXAMPLE A.2

a) A mixture of (±)- 1,1-dimethyl 7-(phenylmethyl)-1,4-dioxa-8-azaspiro[4,5]decane-8-carboxylate (33.34 g; prepared according to the method described in EP-A-532,456) in HCl (6 N; 250 ml) was stirred at 70° C. for 1 hour and 30 minutes. The mixture was cooled, alkalized with NaOH while cooling to 25° C., and extracted with DCM (100 ml). The organic layer was separated and the aqueous layer was extracted with CH₂Cl₂. Triethylamine (20.2 g), followed by 3,5-bis(trifluoromethyl)benzoyl chloride (27.7 g) dissolved in a little DCM were added and the mixture was stirred for 2 hours. The mixture was extracted with water, and the layers were separated. The organic layer. was dried, filtered and the solvent evaporated. The residue was crystallized from DIPE, the precipitate was filtered off and dried, yielding 18.34 g product. The mother layer was evaporated and the residue was crystallized from DIPE. The precipitate was filtered off and dried, yielding 6.51 g of product. The two fractions were put together and taken up in water and DCM, NaOH was added and the mixture was extracted. The organic layer was dried, filtered and the solvent evaporated, yielding 16.14 g (38%) of (±)- 1-[3,5-bis(trifluoromethyl)benzoyl]-2-(phenylmethyl)-4-piperidinone (intermediate 3; mp.102.5° C).

### EXAMPLE A.3

A mixture of pyrrolidine (2.13 g) and triethylamine (6.06 g) in DCM (100 ml) was stirred at −10° C. 2-chloro-2-phenylacetylchloride (5.67 g) was added slowly and dropwise. The mixture was allowed to warm to RT and was then stirred overnight. The mixture was extracted with water and K₂CO₃. The separated organic layer was dried, filtered and the solvent was evaporated. The residue was crystallized from DIPE and the precipitate was filtered off and dried, yielding 3.25 g (48%) of fraction 1. The mother layer was separated and the solvent was evaporated. The residue was crystallized from DIPE and the precipitate was filtered off and dried, yielding 0.29 g (5%) of fraction 2. Both fractions were combined, thus yielding 3.54 g (53%) of (±)- 1-(2-chloro-2-phenylacetyl)pyrrolidine (intermediate 4; mp. 88.5° C.).

### EXAMPLE A.4

Sodium hydride (2 g) was added portionwise to a solution of 3,5-dimethylphenol (6.1 g) in DMF (50 ml). The mixture was stirred for 30 minutes and added dropwise at a temperature below 30° C. to a solution of 2-chloro-2-phenylacetylchloride (9.45 g) in DMF (50 ml). The mixture was stirred overnight, decomposed with water (5 ml) and the solvent was evaporated. Water was added and the mixture was extracted with DCM. The separated organic layer was dried, filtered and the solvent was evaporated. The residue was purified over silica gel on a glass filter (eluent:hexane/DIPE 100/0, 98/2 and 95/5). The pure fractions were collected and the solvent was evaporated [residue; yielding 10.82 g (79%)]. A small amount of the obtained residue was crystallized from DIPE, the precipitate was filtered off and the solvent was evaporated, yielding 1 g of (±)-3,5-dimethylphenyl α-chlorobenzeneacetate (intermediate 5; mp. 79.0° C.).

### EXAMPLE A.5

a) A mixture of (±)-1-[3,5-bis(trifluoromethyl)benzoyl]-2-(phenylmethyl)-4-(1-piperazinyl)piperidine (0.0127 mol), chloroacetonitrile (0.013 mol) and sodium carbonate (0.013 mol) in methylisobutyl keton (100 ml) was stirred and refluxed. The mixture was cooled and water was added. The organic layer was separated, dried, filtered and the solvent was evaporated. The residue was purified over silica gel on a glass filter (eluent: CH₂Cl₂/CH₃OH 100/0, 99.5/0.5 and 99/1). The pure fractions were collected and the solvent was evaporated, yielding 3.64g (53%) of (±)-cis-1-[3,5-bis-(trifluoromethyl)benzoyl]-4-[4-(cyanomethyl)-1-piperazinyl]-2-(phenylmethyl)-piperidine (intermediate 6).
b) A mixture of intermediate 6 (0.0067 mol) in THF (150 ml) was hydrogenated at 20° C. with Raney Nickel (1 g) as a catalyst. After uptake of hydrogen, the catalyst was filtered off and the filtrate was evaporated, yielding 3.77 g of (±)-cis-4-[4-(2-aminoethyl)-1-piperazinyl]-1-[3,5-(trifluoromethyl)benzoyl]-2-(phenylmethyl)-piperidine (intermediate 7).

### EXAMPLE A.6

A mixture of 1-(phenylmethyl)-4-piperidinone (0.2 mol) and 1-methylpiperazine (0.2 mol) in methanol (500 ml) was hydrogenated for 8 hours with palladium on activated carbon (10%, 2.5 g) as a catalyst. After uptake of hydrogen, the catalyst was filtered off and the filtrate was evaporated. A mixture of di-tert -butyl dicarbonate (0.2 mol) in THF (500 ml) was added to the residue and hydrogenated again with palladium on activated carbon (10%, 2.5 g) as a catalyst. After uptake of hydrogen, the catalyst was filtered off and the filtrate was evaporated. The residue was purified over silica gel on a glass filter (eluent: CH₂Cl₂/CH₃OH 95/5). The pure fractions were collected and the solvent was evaporated, yielding 45.3 g (80%) of 1,1-dimethylethyl 4-(4-methyl- 1-piperazinyl)- 1-piperidinecarboxylate (intermediate 8).

### EXAMPLE A.7

a) A mixture of 4-methoxypyridine (0.4 mol) in THF (1000 ml) was stirred and cooled in a 2-propanol/CO₂ bath. Ethyl chloroformate (0.4 mol) was added dropwise and the mixture was stirred for 3 hours while cooling (mixture I). In another round-bottom flask, the Grignard-reagent was prepared: Mg (0.44 mol) was stirred in a small amount of (C₂H₅)₂O. Some 12 was added. A small amount of 1,2-dichloro-4-(chloromethyl)-benzene was added. Then, 1,2-dichloro-4-(chloromethyl)benzene (0.4 mol) in (C₂H₅)₂O (600 ml) was added dropwise at reflux temperature. The mixture was stirred for one hour (mixture II). The Grignard-reagent was decanted off, added to mixture I at <−40° C., and the resulting reaction mixture was stirred, allowing the temperature to reach RT. The reaction mixture was stirred for one hour at RT. HCl (10%, 800 ml) was added and the mixture was stirred for 30 minutes, then CH₂Cl₂ was added. The organic layer was separated, dried, filtered and the solvent evaporated, yielding 57.8 g (44%) of (±)-ethyl 6-[(3,4-dichlorophenyl)methyl]-1,2,3,4-tetrahydro-4-oxo- 1-pyridine-carboxylate (intermediate 9).
b) Intermediate 9 (0.176 mol) in THF (880 ml) was stirred under a N₂ flow, and cooled to −78° C. L-selectride (0.264 mol) was added dropwise at −78° C. The reaction mixture was stirred for 1 hour, then poured out into water. DIPE was added. The organic layer was separated, washed with an aqueous NaHCO₃ solution, with an aqueous NaCl solution, dried, filtered and the solvent was evaporated. The residue was purified by column chromatography over silica gel (eluent: CH₂Cl₂/CH₃OH 90/10). The desired fractions were collected and the solvent was evaporated, yielding 20.2 g (34.8%) of (±)-ethyl 2-[(3,4-dichlorophenyl)methyl]-4-oxo-1-piperidinecarboxylate (intermediate 10).
c) Titanium(IV)isopropoxide (0.0269 mol) was added to a mixture of intermediate 10 (0.0224 mol) and intermediate 10 (0.0224 mol) in DCM (11 ml). The mixture was stirred at RT for 3 hours. Sodium cyanoborohydride (0.0224 mol) and then ethanol (10 ml) were added. The mixture was stirred at RT for 48 hours. Water was added and the mixture was stirred. CH₂Cl₂ was added and the mixture was stirred. The organic layer was separated, dried, filtered and the solvent was evaporated. The residue was purified by HPLC over silica gel (eluent: CH₂Cl₂/CH₃OH 100/0 and 98/2). The pure fractions were collected and the solvent was evaporated. The residue was purified by reversed phase chromatography (eluent: NH₄OAc(0.5% in H₂O)/CH₃OH 20/80). Two pure fractions were collected and their solvents were evaporated. The residue was dried and ground, yielding 2 g (1 6%) of (±)-ethyl trans-2-[(3,4-dichlorophenyl)methyl]-4-[4-[2-[(2,6-dimethylphenyl)amino]-2-oxoethyl]-1-piperazinyl]- 1-piperidinecarboxylate (intermediate 11) and 3.5g (28%) of (±)-ethyl cis-2-[(3,4-dichlorophenyl)methyl]-4-[4-[2-[(2,6-dimethylphenyl)amino]-2-oxoethyl]-1-piperazinyl]-1-piperidinecarboxylate (intermediate 12).
d) A mixture of intermediate 11 (0.0034 mol) and potassium hydroxide (0.034 mol) in 2-propanol (150 ml) was stirred and refluxed for 4 days. The solvent was evaporated. The residue was taken up in CH₂Cl_{2/}water. The organic layer was separated, dried, filtered and the solvent was evaporated. The residue was purified by column chromatography over silica gel (eluent: CH₂Cl₂/(CH₃OH/NH₃) 95/5). The pure fractions were collected and the solvent was evaporated, yielding 0.5 g (30%) of (±)-trans-4-[2-[(3,4-dichlorophenyl)methyl]-4-piperidinyl]-N-(2,6-dimethylphenyl)-1-piperazineacetamide (intermediate 13).

### EXAMPLE A.8

a) Sec-butyllithium (0.066 mol) was added to a mixture of 1,1-dimethylethyl 1,4-dioxo-8-azaspiro[4.5]-8-carboxylate (0.06 mol) in N,N,N′N′-tetramethylethylenediamine (22.6 ml) and (C₂H₅)₂O (100 ml). The mixture was stirred at −70° C. for 3 hours. 3,5-difluorobenzaldehyde (0.07 mol) was added dropwise at −70° C. The mixture was allowed to warm to RT. Water (50 ml) and DIPE were added. The aqueous layer was separated and extracted with CH₂Cl₂. The combined organic layer was dried, filtered and the solvent was evaporated. Toluene was added and evaporated again, yielding 23 g of (±)- 1,1-dimethylethyl 7-[(3,5-difluorophenyl)hydroxymethyl]-1,4-dioxo-8-azaspiro[4.5]-8-carboxylate (intermediate 14)
b) A mixture of intermediate 14 (0.06 mol) and 2-methyl-2-propanol, potassium salt (0.72 g) in toluene (110 ml) was stirred and refluxed for 2 hours. The solvent was evaporated. The residue was stirred in petroleum ether and a small amount of water, and decanted. The residue was dissolved in CH₂Cl₂, dried, filtered and the solvent was evaporated. The residue was purified by column chromatography over silica gel (eluent: CH₂Cl₂/CH₃OH 100/0, 99/1 and 98/2). Two pure fractions were collected and their solvents were evaporated, yielding 9.2 g (49%) of (±)-3-(3,5-difluorophenyl)-tetrahydrospiro[1,3-dioxolan-2,5′(3′H)- 1H-oxazolo[3,4-a]pyridin]-1-one (intermediate 15)
c) A mixture of intermediate 15 (0.03 mol) in methanol (250 ml) was hydrogenated at 50° C. with palladium on activated carbon (10%, 2 g) as a catalyst. After uptake of hydrogen, the catalyst was filtered off and the filtrate was evaporated. The residue was purified over silica gel on a glass filter (eluent: CH₂Cl₂/CH₃OH 100/0, 98/2 and 95/5 and CH₂Cl₂/(CH₃OH/NH₃) 95/5). The desired fractions were collected and the solvent was evaporated, yielding 1.9 g (39%) of (±)-7-[(3,5-difluorophenyl)methyl]-1,4-dioxo-8-azaspiro[4.5]decane (intermediate 16).
d) A mixture of intermediate 16 (0.012 mol) in HCl 6N (30 ml) was stirred at 75° C. for 2 hours. The mixture was cooled, poured out into ice and a NaOH solution and extracted with CH₂Cl₂. The organic layer was separated, dried and filtered, yielding 2.7 g of (±)-2-[(3,4-difluorophenyl)methyl]-4-piperidinone (intermediate 17).
e) A mixture of 3,5-trifluoromethylbenzoyl chloride (0.012 mol) in a small amount of CH₂Cl₂ was added dropwise to a stirred mixture of intermediate 17 (0.012 mol) and N,N-diethylethanamine (0.024 mol). The mixture was stirred at RT for 1 hour and water was added. The organic layer was separated, dried, filtered and the solvent was evaporated. The residue was purified over silica gel on a glass filter (eluent: CH₂Cl₂/CH₃OH 100/0 and 99.5/0.5). The pure fractions were collected and the solvent was evaporated, yielding 2.7g (48%) of (±)-1-[3,5-bis(trifluoromethyl)benzoyl]-2-[(3,5-difluorophenyl)methyl]-4-piperidinone (intermediate 18).

### EXAMPLE A.9

Sec-butyllithium (0.63 mol) was added at −78° C. under N₂ flow to a solution of 1,1-dimethylethyl 1,4-dioxo-8-azaspiro[4.5]-8-carboxylate (0.57 mol) and N,N,N′N′-tetramethylethylenediamine (1.14 mol) in (C₂H₅)₂O (1000 ml). One hour after complete addition, a mixture of 3-(trifluoromethyl)benzaldehyde (0.57 mol) in (C₂H₅)₂O (200 ml) was added. The mixture was allowed) to warm to RT and then stirred at RT for 16 hours. The solvent was evaporated. A mixture of 2-methyl-2-propanol, potassium salt (0.2 mol) in toluene (500 ml) was added. The mixture was stirred. at 80° C. for 5 hours. The solvent was evaporated. The residue was heated with a saturated NH₄Cl solution and extracted with CH₂Cl₂. The organic layer was separated, dried, filtered and the solvent was evaporated. The residue was suspended in DIPE, filtered off and dried. This fraction was dissolved in CH₃OH (250 ml) and the mixture was hydrogenated with palladium on activated carbon (10%, 3 g) as a catalyst. After uptake of hydrogen, the catalyst was filtered off and the filtrate was evaporated. The residue was purified over silica gel on a glass filter (eluent: CH₂Cl₂/CH₃OH 95/5). The pure fractions were collected and the solvent was evaporated. This fraction was dissolved in HCl (6 N, 100 ml) and CH₃OH (100 ml) and the mixture was stirred at 50° C. for 8 hours. The organic solvent was evaporated. The concentrate was washed with a saturated K₂CO₃ solution and extracted with CH₂Cl₂. The organic layer was separated, dried, filtered and the solvent was evaporated. The residue was purified over silica gel on a glass filter (eluent: CH₂Cl₂/CH₃OH 95/5). The pure fractions were collected and the solvent was evaporated, yielding 48.5 g (70%) of (±)-2-[[4-(trifluoromethyl)phenyl]methyl]-4-piperidinone (intermediate 19).

### EXAMPLE A.10

a) A mixture of ethyl β-oxobenzenebutanoate (0.5 mol) and benzenemethanamine (0.5 mol) in toluene (500 ml) was hydrogenated at 120° C. (pressure=100 kg) overnight in the presence of Cu₂Cr₂O₅ (5 g) and CaO (10 g). After uptake of hydrogen, the catalyst was filtered off and the filtrate was evaporated, yielding 29.7 g of (±)-ethyl N,2-bis(phenylmethyl)-β-alanine (intermediate 20).
b) Ethyl chloroacetate (0.3 mol) was added to a mixture of intermediate 20 (0.2 mol) in DMF (250 ml). The mixture was stirred and triethylamine (0.4 mol) was added. The mixture was stirred at 60° C. overnight. The solvent was evaporated and the residue was taken up in water/CH₂Cl₂. The organic layer was separated, dried, filtered and the solvent was evaporated, yielding 76.6 g of (±)-ethyl 3-[(2-ethoxy-2-oxoethyl)(phenylmethyl)amino]benzenebutanoate (intermediate 21).
c) Intermediate 21 (0.2 mol) was heated to 80° C. under N₂ flow. NaOCH₃ (44 g) was added. The mixture was stirred at 80° C. for 30 minutes. The solvent was evaporated and water (170 ml) and HCl (6 N, 60 ml) were added. The mixture was stirred and refluxed for 1 hour, then cooled, alkalized with NaOH and extracted with CH₂Cl₂. The organic layer was separated, washed with water and a saturated NaCl solution, dried, filtered and the solvent was evaporated. The residue was purified by column chromatography over silica gel (eluent: CH₂Cl₂/CH₃CN 100/0 to 96/4). The pure fractions were collected and the solvent was evaporated, yielding 7.8 g of (±)-1,5-bis(phenylmethyl)-3-pyrrolidinone (intermediate 22).
d) A mixture of intermediate 22 (0.027 mol) and CH₃SO₃H (0.03 mol) in THF (200 ml) was hydrogenated with palladium on activated carbon (10%, 2 g) as a catalyst. After uptake of hydrogen, the catalyst was filtered off, yielding (±)-5-(phenylmethyl)-3-pyrrolidinone methanesulfonate(1:1) (intermediate 23).
e) 3,5-di(trifluoromethyl)benzoyl chloride (0.03 mol) was added to intermediate 23 (0.027 mol). The mixture was stirred and triethylamine (0.1 mol) was added. The mixture was stirred at RT for 18 hours and then washed with water, NaOH and a saturated NaCl solution. The organic layer was separated, washed with a saturated NaCl solution, dried, filtered and the solvent was evaporated. The residue was purified by column chromatography over silica gel (eluent: CH₂Cl₂/CH₃OH 98/2). The pure fractions were collected and the solvent was evaporated, yielding 1.4 g of (±)-1-[3,5-bis(trifluoromethyl)benzoyl]-5-(phenylmethyl)-3-pyrrolidinone (intermediate 24).

### B. Preparation of the Compounds of Formula (I)

### EXAMPLE B.1

a) Titanium(IV)isopropoxide (16.5 g) was added to a mixture of intermediate 3 (21.5 g) and 1-(phenylmethyl)piperazine (8.81 g) in DCM (35 ml). The mixture was stirred for 3 hours at RT. Sodium cyanoborohydride (2.85 g) and ethanol (70 ml) were added and the resulting reaction mixture was stirred overnight at RT. Water (5 ml) and DCM were added. The biphasic mixture was filtered over dicalite, and the filter residue was washed with DCM. The organic layer was separated, dried, filtered and the solvent was evaporated. The residue was crystallized from CH₃CN and the precipitate was filtered off and dried, yielding 7.93 g (26.9%) of (±)-cis-1-[3,5-bis(trifluoromethyl)benzoyl]-2-(phenylmethyl)-4-[4-(phenylmethyl)-1-piperazinyl]piperidine (compound 16; mp. 143.8° C.).
b) The mother liquor was concentrated and the residue was purified by column chromatography over silica gel (eluent: CH₂Cl₂/CH₃OH 100/0, then 99/1, 98/2, 97/3). The desired fractions ((A) and (B)) were collected and their solvent was evaporated. The A-isomer was crystallized from CH₃CN, filtered off and dried, yielding 1.11 g (4%) of compound 16. The pure fractions of the B-isomer were concentrated, yielding 5.9 g (20%) of (±)-trans-1-[3,5-bis(trifluoromethyl)benzoyl]-2-(phenylmethyl)-4-[4-(phenylmethyl)-1-piperazinyl]piperidine. The impure fractions of the B-isomer were collected and the solvent was evaporated. The residue was converted into the fumaric acid salt (1:2) in ethanol. The precipitate was filtered off and dried, yielding 1.89 g (i)-trans-1-[3,5-bis(trifluoromethyl)benzoyl]-2-(phenylmethyl)-4-[4-(phenylmethyl)- 1-piperazinyl]piperidine (E)-2-butenedioate(1:2) (compound 17; mp. 240.3° C.).

### EXAMPLE B.2

A mixture of compound 16 (8.4 g) in methanol (250 ml) was hydrogenated at 50° C. with palladium on activated carbon (10%) (2 g) as a catalyst. After uptake of H₂, the catalyst was filtered off and the filtrate was evaporated, yielding 7 g (100%) of (±)-1-[3,5-bis(trifluoromethyl)benzoyl]-2-(phenylmethyl)-4-(1-piperazinyl)piperidine (compound 15).

### EXAMPLE B.3

a) Titanium(IV)isopropoxide (13.2 g) was added to a mixture of intermediate 3 (17.16 g) and N-(2,6-dimethylphenyl)-1-piperazineacetamide (9.88 g) in DCM (20 ml). This mixture was stirred for 3 hours at RT. Sodium cyanoborohydride (2.52 g) in ethanol (20 ml) was added and the resulting reaction mixture was stirred overnight at RT. Water (10 ml) was added and the reaction mixture was extracted with DCM (800 ml). The organic layer was separated, dried, filtered and the solvent was evaporated. The residue was taken up into water and this mixture was extracted with DCM. The separated organic layer was dried, filtered, and the solvent evaporated. The residue was pre-purified by column chromatography over silica gel (eluent: CH₂Cl₂/CH₃OH 97/3). The desired fractions were collected and the solvent was evaporated, giving 4 g of the trans-racemate. Resolution was obtained by purification over stationary phase Chiralcel OD (eluent: CH₃OH 100%). Two desired trans-fraction groups were collected and their solvent was evaporated, yielding 1.75 g fraction 1 and 2 g fraction 2. Fraction 1 was dissolved in DCM, filtered and the filtrate was evaporated. The residue was dried, yielding 1.55 g (6%) (−)-(A)-trans-4-[1-[3,5-bis(trifluoromethyl)benzoyl]-2-(phenyl-methyl)-4-piperidinyl]-N-(2,6-dimethylphenyl)-1-piperazine acetamide (compound 26; mp. 97.4° C.; [α]_{D}^{°}=−5.81° (c.=1% in DMF)). Fraction 2 was dissolved in DCM, filtered and the filtrate was evaporated. The residue was dried, yielding 1.70 g (6%) (+)-(B)-trans-4-[1-[3,5-bis(trifluoromethyl)benzoyl]-2-(phenylmethyl)-4-piperidinyl]-N-(2,6-dimethylphenyl)-1-piperazine acetamide (compound 27; mp. 96.8° C.; [α]_{D}²⁰=+5.71° (c=1% in DMF)).
b) Compound 27 was dissolved in warm 2-propanol and converted into the (L)-malic acid salt with a solution of (L)-malic acid in 2-propanol. The mixture was stirred for 2 hours and the precipitate was filtered off and dried, yielding (+)-(B)-trans-4-[1-[3,5-bis(trifluoromethyl)benzoyl]-2-(phenylmethyl)-4-piperidinyl]-N-(2,6-dimethylphenyl)-1-piperazine acetamide (L)-malic acid (1:1) (compound 95).

### EXAMPLE B.4

A mixture of compound 15 (2.5 g), intermediate 5 (1.65 g) and sodium carbonate (0.64 g) in methylisobutylketon (50 ml) was stirred and refluxed for 3 hours. The reaction mixture was washed and the separated organic layer was dried, filtered and the solvent was evaporated. The residue was purified over silica gel on a glass filter (eluent: CH₂Cl₂/CH₃OH 100/0 and 99.5/0.5). The pure fractions were collected and the solvent was evaporated, yielding 1.59 g (43%) of (±)-3,5-dimethylphenyl cis-4-[1-[3,5-bis(trifluoromethyl)benzoyl]-2-(phenylmethyl)-4-piperidinyl]-α-phenyl- 1-piperazine-acetate (compound 43; mp. 88.1° C.).

### EXAMPLE B.5

A mixture of intermediate 2 (3.2 g), 1-(diphenylmethyl)piperazine (2.5 g) and aluminum tributoxide (2 g) in toluene (250 ml) was hydrogenated for 48 hours at 50° C., with palladium on activated carbon (10%; 2 g) as a catalyst in the presence of thiophene (4% solution; 1 ml). After uptake of hydrogen (1 equiv), the catalyst was filtered off and the filtrate was evaporated. The residue was purified by high-performance liquid chromatography over silica gel (eluent: CH₂Cl₂/CH₃OH 100/0, upgrading to 90/10). Two pure fractions were collected and their solvent was evaporated, resulting in residue 1 and residue 2. Residue 1 was suspended in DIPE. The precipitate was filtered off and dried, yielding 0.94 g (17%) of (±)-cis-1-(dimethylbenzoyl)-4-[4-(diphenylmethyl)-1-piperazinyl]-2-(phenylmethyl)piperidine (compound 12; mp. 100.8° C.). Residue 2 was dried, yielding 0.2 g (3.6%) of (±)-trans-1-(dimethylbenzoyl)-4-[4-(diphenylmethyl)-1-piperazinyl]-2-(phenylmethyl)piperidine (compound 13).

### EXAMPLE B.6

A mixture of compound 15 (0.005 mol) and 1,2-epoxyethylbenzene (0.006 mol) in methanol (50 ml) was stirred at RT for 1 hour. The mixture was stirred and refluxed for 3 hours. The solvent was evaporated and the residue was purified over silica gel on a glass filter (eluent: CH₂Cl₂/CH₃OH 100/0, 99/1 and 98/2). The pure fractions were collected and the solvent was evaporated. The residue was purified by HPLC over silica gel (eluent: CH₂Cl₂/CH₃OH 98/2 to 95/5). Two pure fractions were collected and their solvents were evaporated. Each residue was dried, yielding 0.7 g (23%) of (±)-cis- 1-[3,5-bis(trifluoromethyl)benzoyl]-4-[4-(2-hydroxy-2-phenylethyl)-1-piperazinyl]-2-(phenylmethyl)piperidine (compound 60) and 0.23 g (7%) of (±)-cis- 1-[3,5-bis(trifluoromethyl)benzoyl]-4-[4-(2-hydroxy- 1-phenylethyl)-1-piperazinyl]-2-(phenylmethyl)piperidine (compound 61).

### EXAMPLE B.7

Compound 15 (0.005 mol), 2-chloro-1-[(2-methyl-5-oxazolyl)methyl]-1H-benzimidazole (0.005 mol) and Cupper (0.005 mol) were stirred at 140° C. for 2 hours. The mixture was cooled, dissolved in CH₂Cl₂, filtered and washed with CH₂Cl₂ and a diluted NH₄OH solution. The organic layer was separated, washed with a diluted NH₄OH solution, dried, filtered and the solvent was evaporated. The residue was purified over silica gel on a glass filter (eluent: CH₂Cl₂/CH₃OH 100/0, 99.5/0.5, 99/1, 98.5/1.5 and 98/2). The pure fractions were collected and the solvent was evaporated. The residue was dried, yielding 1.42 g (40%) (±)-cis-1-[3,5-bis(trifluoromethyl)-benzoyl]-4-[4-[1-[(2-methyl-5-oxazolyl)methyl]-1H-benzimidazol-2-yl]- 1-piperazinyl]-2-(phenylmethyl)piperidine (compound 70).

### EXAMPLE B.8

A mixture of intermediate 7 (0.0033 mol) and 3,5-dimethylbenzoyl chloride (0.0035 mol) in DCM (50 ml) was stirred at RT for 15 minutes. Triethylamine (0.007 mol) was added and the mixture was stirred at RT for 1 hour. Water was added. The organic layer was separated, dried, filtered and the solvent was evaporated. The residue was converted into the fumaric acid salt (1:1) with 2-propanol. The precipitate was filtered off and dried. The residue was converted into the free base with NaOH. The precipitate was filtered off and dried. The residue was purified over silica gel on a glass filter (eluent: CH₂Cl₂/CH₃OH 100/0, 99.5/0.5, 99/1, 98/2 and 97/3). The pure fractions were collected and the solvent was evaporated. The residue was dried, yielding 0.8 g (36%) (±)-cis-N-[2-[4-[1-[3,5-bis(trifluoromethyl)benzoyl]-2-(phenylmethyl)-4-piperidinyl]- 1-piperazinyl]ethyl]-3,5-dimethylbenzamide (compound 116).

### EXAMPLE B.9

A mixture of compound 74, prepared according to example B.4, (0.004 mol) in methanol (150 ml) was hydrogenated at 50° C. with palladium on activated carbon (10%; 1 g) as a catalyst in the presence of thiophene (4% solution, 1 ml). After uptake of hydrogen, the catalyst was filtered off and the filtrate was evaporated. The residue was crystallized from DIPE. The precipitate was filtered off, washed with DIPE and dried. This fraction was dissolved in toluene. The mixture was filtered and the solvent was evaporated. The residue was suspended in DIPE. The precipitate was filtered off and dried. This fraction was converted into the fumaric acid salt (1:2) with a warm solution of fumaric acid (0.52g) in ethanol. The mixture was stirred for 6 hours. The precipitate was filtered off and dried, yielding 0.91 g (25%) of (±)-cis-N-(4-amino-2,6-dimethylphenyl)-4-[1-[3,5-(trifluoromethyl)benzoyl]-2-(phenylmethyl)-4-piperidinyl]-1-piperazineacetamide (E)-2-butenedioate(1:2) (compound 129).

### EXAMPLE B.10

Sec-butyllithium (0.055 mol) was added at −78° C. under N₂ flow to a solution of 1,1-dimethylethyl 4-(4-methyl-1-piperazinyl)-1-piperidinecarboxylate (0.05 mol) and N,N,N,N′-tetramethylethylenediamine (0.1 mol) in (C₂H₅)₂O (50 ml). 2 hours after complete addition, a mixture of benzaldehyde (0.05 mol) in (C₂H₅)₂O (50 ml) was added. The mixture was allowed to warm to RT and then stirred at 25° C. for 16 hours. The solvent was evaporated and the residue was washed with a saturated NH₄Cl solution and extracted with CH₂Cl₂. The organic layer was separated, dried, filtered and the solvent was evaporated. A solution of 2-methyl-2-propanol, potassium salt (0.02 mol) in toluene (100 ml) was added to this fraction and the mixture was stirred at 100° C. for 2 hours. The solvent was evaporated. The residue was washed with a saturated NH₄Cl solution, extracted with CH₂Cl₂ and decanted. The organic layer was dried, filtered and the solvent was evaporated. The residue was purified over silica gel on a glass filter (eluent: CH₂Cl₂/CH₃OH 95/5). The pure fractions were collected and the solvent was evaporated. This fraction was dissolved in methanol (150 ml) and hydrogenated with palladium on activated carbon (10%, 3 g) as a catalyst. After uptake of hydrogen, the catalyst was filtered off and the filtrate was evaporated. The residue was purified over silica gel on a glass filter (eluent: CH₂Cl₂/CH₃OH 95/5). The pure fractions were collected and the solvent was evaporated. This fraction was dissolved in DCM (20 ml) and Triethylamine (2 ml). 3,5-di(trifluoromethyl)benzoyl chloride (0.0087 mol) was added at 0° C. 1 hour after complete addition, water was added and the mixture was extracted with CH₂Cl₂. The organic layer was separated, dried, filtered and the solvent was evaporated. The residue was purified over silica gel on a glass filter (eluent: CH₂Cl₂/CH₃OH 95/5). The pure fractions were collected and the solvent was evaporated. This fraction was converted into the (E)-2-butenedioic acid salt (1:2) with ethanol. The precipitate was filtered off and dried, yielding 4.7 g (74%) of (±)-cis-1-[3,5-bis(trifluoromethyl)benzoyl]-4-(4-methyl- 1-piperazinyl)-2-(phenylmethyl)-piperidine (E)-2-butenedioate(1:2) (compound 130).

### EXAMPLE B.11

A mixture of compound 15 (0.005 mol), N-[2-(3,4-dichlorophenyl)-4-[(methylsulfonyl)-oxy]butyl]-N-methyl benzamide (0.0055 mol) and NaHCO₃ (0.0055 mol) in ethanol (50 ml) was stirred and refluxed for 6 hours. The solvent was evaporated, the residue was taken up in water and extracted with CH₂Cl₂. The organic layer was separated, dried, filtered and the solvent was evaporated. The residue was purified over silica gel on a glass filter (eluent: CH₂Cl₂/CH₃OH 100/0, 99/1, 98/2 and 97/3). The pure fractions were collected and the solvent was evaporated. The residue was converted into the fumaric acid salt (1:2) with ethanol. The precipitate was filtered off and dried, yielding 1.42 g (27%) of (±)-cis-N-[4-[4-[1-[3,5-bis(trifluoromethyl)benzoyl]-2-(phenylmethyl)-4-piperidinyl]-1-piperazinyl]-2-(3,4-dichlorophenyl)butyl]-N-methyl-benzamide (E)-2-butenedioate(1:2) (compound 93).

### EXAMPLE B. 12

A mixture of (±)-1-[3,5-bis(trifluoromethyl)benzoyl]-2-[(3,5-difluorophenyl)methyl]-4-piperidinone (0.0058 mol), N-(2,6-dimethylphenyl)-1-piperazineacetamide (0.0058 mol) and titanium(IV)isopropoxide (0.0064 mol) in 2-propanol (5 ml) was stirred at RT overnight. NaBH₄ (0.0116 mol) and ethanol (15 ml) were added. The mixture was stirred for 2 days. Water (5 ml) was added and the mixture was stirred for 10 minutes. CH₂Cl₂ (200 ml) was added. The organic layer was separated, dried, filtered and the solvent was evaporated. This fraction was purified by HPLC over silica gel (eluent: CH₂Cl₂/CH₃OH 98/2 to 90/10 over a 30-minute period). Two pure fractions (F1 and F2) were collected and their solvents were evaporated. F1 was purified by column chromatography over RP18 (eluent: NH₄OAc (0.5% in H₂O)/CH₃CN 40/60). The pure fractions were collected and the solvent was evaporated. The residue was dried, yielding 0.33 g (8%) of (±)-cis-4-[1-[3,5-bis(trifluoromethyl)benzoyl]-2-[(3,5-difluorophenyl)-methyl]-4-piperidinyl]-N-(2,6-dimethylphenyl)-1-piperazineacetamide (compound 132). F2 was purified by column chromatography over silica gel (eluent: CH₂Cl₂/CH₃OH 100/0 to 92/8 over a 30-minute period). The pure fractions were collected and the solvent was evaporated. The residue was dissolved in CH₂Cl₂, filtered and the solvent was evaporated. The residue was dried, yielding 0.24 g (6%) of (±)-trans-4-[1-[3,5-bis-(trifluoromethyl)benzoyl]-2-[(3,5-difluorophenyl)methyl]-4-piperidinyl]-N-(2,6-dimethylphenyl)-1-piperazineacetamide (compound 133).

### EXAMPLE B.13

3,5 di(trifluormethyl)benzoyl chloride (0.0011 mol) was added to a mixture of (±)-trans-4-[2-[(3,4-dichlorophenyl)methyl]-4-piperidinyl]-N-(2,6-dimethylphenyl)-1-piperazineacetamide (0.001 mol) in DCM (20 ml). The mixture was stirred for 5 minutes. Triethylamine (2 ml) was added. The mixture was stirred at RT for 3 hours, washed with a diluted NaOH solution and with water, and then dried. The solvent was evaporated. The residue was purified by column chromatography over silica gel (eluent: CH₂Cl₂/CH₃OH 96/4). The pure fractions were collected and the solvent was evaporated. The residue was crystallized from CH₃CN. The precipitate was filtered off and dried, yielding 0.32 g (44%) of (±)-trans-4-[1-[3,5-bis(trifluoromethyl)benzoyl]-2-[(3,4-dichlorophenyl)methyl]-4-piperidinyl] -N-(2,6-dimethylphenyl)- 1-piperazine-acetamide (compound 139).

### EXAMPLE B.14

A mixture of compound 15 (0.01 mol) and imidazo[1,2-a]pyridin-2-carboxaldehyde (0.01 mol) in methanol (250 ml) was hydrogenated at RT overnight with palladium on activated carbon (10%, 2 g) as a catalyst in the presence of thiophene (4% solution, 2 ml). After uptake of hydrogen, the catalyst was filtered off and the filtrate was evaporated. The residue was purified over silica gel on a glass filter (eluent: CH₂Cl₂/CH₃OH 100/0, 99/1, 98/2, 97/3 and 96/4). The pure fractions were collected and the solvent was evaporated. The residue was converted into the fumaric acid salt (1:2) from ethanol. The precipitate was filtered off and dried, yielding 2.8 g (32%) of (±)-cis- 1-[3,5-bis(trifluoromethyl)benzoyl]-4-[4-(imidazo[1,2-a]pyridin-2-ylmethyl)-1-piperazinyl]-2-(phenylmethyl)piperidine (E)-2-butenedioate(1:2) (compound 111).

### EXAMPLE B.15

(+)-(B-trans)-4-[1-[3,5-bis(trifluoromethyl)benzoyl]-2-(phenylmethyl)-4-piperidinyl]-N-(2,6-dimethylphenyl)-1-piperazineacetamide (0.003 mol) was dissolved in ethanol (20 ml). A solution of fumaric acid (0.003 mol) in ethanol (15 ml) was added and the mixture was stood for 7 days. The precipitate was filtered off and dried, yielding 1.2 g of (B-trans)-4-[1-[3,5-bis(trifluoromethyl)benzoyl]-2-(phenylmethyl)-4-piperidinyl]-N-(2,6-dimethylphenyl)-1-piperazineacetamide (E)-2-butenedioate(1:1) (compound 128).

### EXAMPLE B.16

A mixture of (±)-1-[3,5-bis(trifluoromethyl)benzoyl]-5-(phenylmethyl)-3-pyrrolidinone (0.0037 mol) and N-(2,6-dimethylphenyl)-l-piperazineacetamide (0.0037 mol) in methanol (150 ml) was hydrogenated at 50° C. with palladium on activated carbon (10%, 1 g) as a catalyst in the presence of tiophene solution (1 ml). After uptake of hydrogen, the catalyst was filtered off and the filtrate was evaporated. The residue was purified by column chromatography over silica gel (eluent: CH₂Cl₂/CH₃OH 95/5). The desired fractions were collected and the solvent was evaporated. The residue was dried and then crystallized from DIPE. The precipitate was filtered off and dried, yielding 0.35 g (15%) of (±)-cis-4-[1-[3,5-bis(trifluoromethyl)benzoyl]-5-(phenylmethyl)-3-pyrrolidinyl]-N-(2,6-dimethylphenyl)-1-piperazineacetamide (compound 131).

### EXAMPLE B.17

(±)-cis-1-(phenylmethyl)-4-[2-(phenylmethyl)-1-piperidinyl]piperazine (0.00043 mol) was added to 3,4-dichlorobenzeneacetic acid (±) 0.0004 mol) and 1-hydroxybenzo-triazole hydrate (0.080 g) in DCM (5 ml). The mixture was stirred and cooled on an ice/ethanol-bath, under N₂ flow. Triethylamine was added dropwise. A solution of 1-(3-dimethylaminopropyl)-3-ethylcarbodiimide hydrochloride (0.120 g) in DCM (5 ml) was added and the reaction mixture was allowed to warm to RT, under N₂. The reaction mixture was stirred overnight. The mixture was diluted with CH₂Cl₂, until a 15-ml total volume was obtained. Then, the compound was isolated and purified by HPLC over silica gel (eluent: CH₂Cl₂ to CH₂Cl₂/CH₃OH 90/10 over 20 minutes at 125 ml/minute). The desired fractions were collected and the solvent was evaporated, yielding 0.020 g of (±)-cis-1-[(3,4-dichlorophenyl)acetyl]-2-(phenylmethyl)-4-[4-(phenylmethyl)- 1-piperazinyl]piperidine (compound 181).

### EXAMPLE B.18

3,5-di(trifluoromethyl)-1-isocyanatobenzene (0.0025 mol) in DCM (10 ml) was added to a mixture of (±)-trans-N-(2,6-dimethylphenyl)-4-[2-(phenylmethyl)-4-piperidinyl]-1-piperazineacetamide (0.0025 mol) in DCM (15 ml). The mixture was stirred at RT overnight. The precipitate was filtered off and dried, yielding 0.66 g (40%) of (±)-trans-4-[1-[[[3,5-bis(trifluoromethyl)phenyl]amino]carbonyl]-2-(phenylmethyl)-4-piperidinyl]-N-(2,6-dimethylphenyl)- 1-piperazineacetamide (compound 143).

### EXAMPLE B.19

A mixture of (±)-1-[3,5-bis(trifluoromethyl)benzoyl]-2-[[3-fluoro-5-(trifluoromethyl) phenyl]methyl]-4-piperidinone (0.01 mol) and N-(2,6-dimethylphenyl)-1-piperazine-acetamide (0.01 mol) in 2-propanol (150 ml) was hydrogenated at 50° C. with platinum on activated carbon (5%; 2 g) as a catalyst in the presence of titanium(IV)isopropoxide (2.84 g) and thiophene solution (1 ml). After uptake of hyrogen, the catalyst was filtered off and the filtrate was evaporated. The residue was taken up in CH₂Cl₂ and H₂O. The organic layer was separated, washed several times with H₂O, dried, filtered over dicalite and the solvent was evaporated. This fraction was purified by HPLC over silica gel (eluent: CH₂Cl₂/CH₃OH 98/2). Two pure fractions were collected and their solvents were evaporated. The residue was dried, yielding 0.72 g (10%) of (±)-cis-4-[1-[3,5-bis(trifluoromethyl)benzoyl]-2-[[3-fluoro-5-(trifluoromethyl)phenyl]methyl]-4-piperidinyl]-N-(2,6-dimethylphenyl)-1-piperazineacetamide (compound 140) and 0.88 g (12%) of (±)-trans-4-[1-[3,5-bis(trifluoromethyl)benzoyl]-2-[[3-fluoro-5-(trifluoro-methyl)phenyl]methyl]-4-piperidinyl]-N-(2,6-dimethylphenyl)-1-piperazineacetamide (compound 141).

### Tables 1 to 4 list compounds of formula (I) that were prepared according to one or more of the foregoing examples (Ex.).

TABLE 1

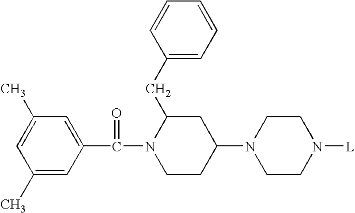

| Co. | | | Physical data |
|---|---|---|---|
| No. | Ex. | —L | (mp = melting point) |
| 1 | 6 | —H | (±)-cis |
| 2 | 6 | —H | (±)-trans |
| 3 | 9 | 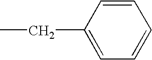 | (±)-cis; mp 196.9° C. |
| 4 | 9 | 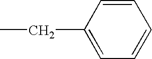 | (±)-trans |
| 5 | 5 | 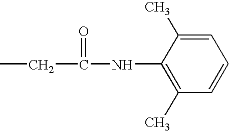 | (±)-cis; mp 94.0° C. |
| 6 | 5 | 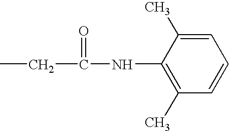 | (±)-trans |
| 7 | 8 | 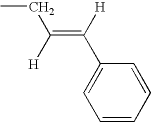 | (±)-cis-(E);mp 201.0° C. |
| 8 | 8 | 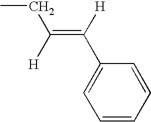 | (±)-trans-(E);mp 210.1° C. |
| 9 | 8 | 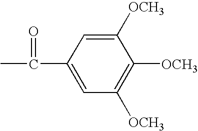 | (±)-cis; mp 92.1° C. |
| 10 | 9 | 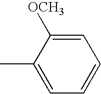 | (±)-cis; mp 72.8° C. |
| 11 | 9 | 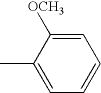 | (±)-trans |
| 12 | 9 | 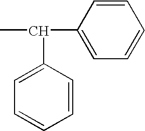 | (±)-cis; mp 100.8° C. |
| 13 | 9 | 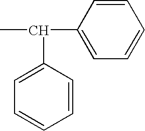 | (±)-trans |

TABLE 2

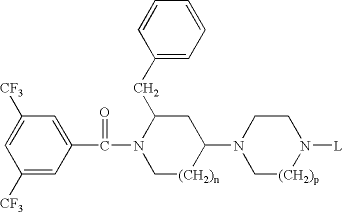

| Co. | | | | | Physical data |
|---|---|---|---|---|---|
| No. | Ex | n | p | —L | (mp = melting point) |
| 14 | B.2 | 1 | 1 | —H | (±)-trans |
| 15 | B.2 | 1 | 1 | —H | (±)-cis |
| 16 | B.1.a | 1 | 1 | 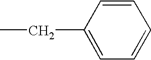 | (±)-cis; mp 143.8° C. |
| 17 | B.1.a + b | 1 | 1 | 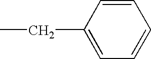 | (±)-trans; mp 240.3° C.;fumaric acid (1:2) |
| 18 | B.1 | 1 | 1 | 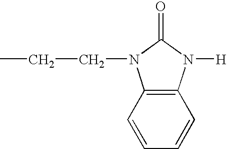 | (±)-cis; mp 120° C. |
| 19 | B.1 | 1 | 1 | 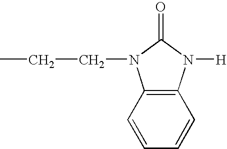 | (±)-trans; mp 150° C. |
| 20 | B.1 | 1 | 1 | 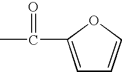 | (±)-cis; mp 70.4° C. |
| 21 | B.1 | 1 | 1 | 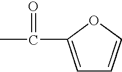 | (±)-trans; mp 169.1° C. |
| 22 | B.1 | 1 | 1 | 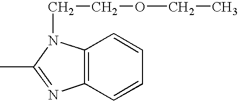 | (±)-trans; mp 173.8° C. |
| 23 | B.1 | 1 | 1 | 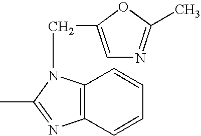 | (±)-cis; mp 93.2° C. |
| 24 | B.1 | 1 | 1 | 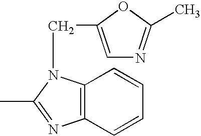 | (±)-trans; mp 100.1° C. |
| 25 | B.1 | 1 | 1 | 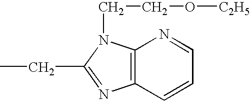 | (±)-trans; mp 75.4° C. |
| 26 | B.1 andB.3 | 1 | 1 | 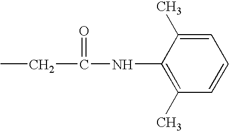 | (−)-(A)-trans; mp 97.4° C.;[α]_{D}²⁰ = −5.81° (c = 1% inDMF) |
| 27 | B.1 andB.3 | 1 | 1 | 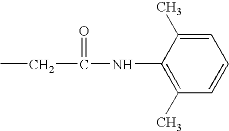 | (+)-(B)-trans; mp 96.8° C.;[α]_{D}²⁰ = +5.71° (c = 1% inDMF) |
| 28 | B.1 | 1 | 1 | 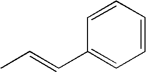 | (±)-cis; (E) |
| 29 | B.1 | 1 | 1 | 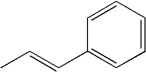 | (±)-trans; (E) |
| 30 | B.1 | 1 | 1 | 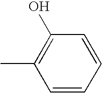 | (±)-trans; mp 185.7° C. |
| 31 | B.1 | 1 | 1 | 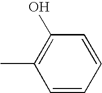 | (±)-cis; mp 77.5° C. |
| 32 | B.1 | 1 | 1 | 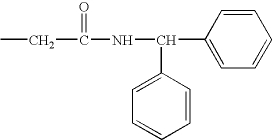 | (±)-cis; mp 183.1° C. |
| 33 | B.1 | 1 | 1 | 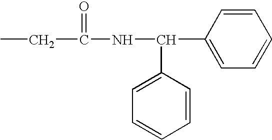 | (±)-trans; mp 115.6° C. |
| 34 | B.1 | 1 | 2 | 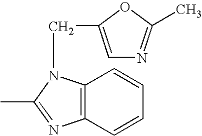 | (±)-trans; mp 120.1° C. |
| 35 | B.1 | 1 | 1 | 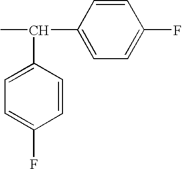 | (±)-cis; mp 150.9° C. |
| 36 | B.1 | 1 | 1 | 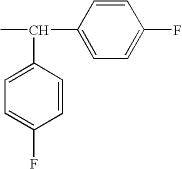 | (±)-trans; mp 120.8° C. |
| 37 | B.4 | 1 | 1 | 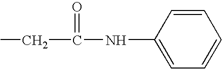 | (±)-cis; mp 85.6° C. |
| 38 | B.4 | 1 | 1 | 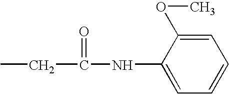 | (±)-trans; mp 170.5° C. |
| 39 | B.4 | 1 | 1 | —CH₂—CH₂—OH | (±)-cis; mp 192.9° C. |
| 40 | B.4 | 1 | 1 | 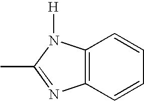 | (±)-trans; mp 240.7° C. |
| 41 | B.4 | 1 | 1 | 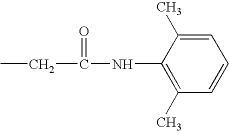 | (±)-(A)-cis; mp 177.3° C.;[α]_{D}²⁰ = +19.88° (c = 1%in methanol) |
| 42 | B.4 | 1 | 1 | 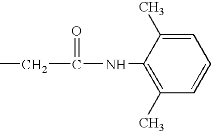 | (−)-(B)-cis; mp 177.3° C.;[α]_{D}²⁰ = −20.34° (c = 1%in methanol) |
| 43 | B.4 | 1 | 1 | 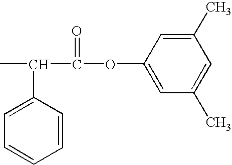 | (±)-cis; mp 88.1° C. |
| 44 | B.4 | 1 | 1 | 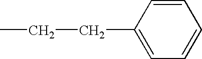 | (±)-cis; mp 227.1° C.;fumaric acid (1:2) |
| 45 | B.4 | 1 | 1 | 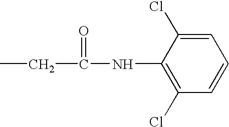 | (±)-trans; mp 200.2° C. |
| 46 | B.4 | 1 | 1 | 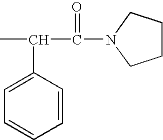 | (±)-trans; mp 105.6° C. |
| 47 | B.4 | 1 | 1 | 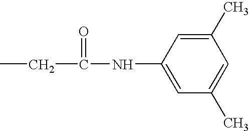 | (±)-cis; mp 89.2° C. |
| 48 | B.1 | 1 | 1 | 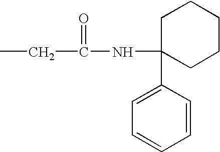 | (±)-trans; mp 89.7° C. |
| 49 | B.1 | 1 | 1 | 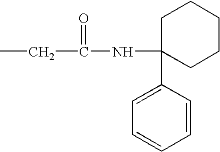 | (±)-cis; mp 135.8° C. |
| 50 | B.4 | 1 | 1 | 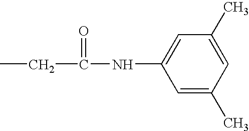 | (±)-trans; mp 140.4° C. |
| 51 | B.4 | 1 | 1 | 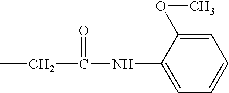 | (±)-cis; mp 173.5° C. |
| 52 | B.4 | 1 | 1 | 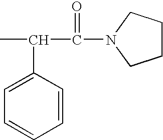 | (±)-cis; mp 101.5° C. |
| 53 | B.4 | 1 | 1 | 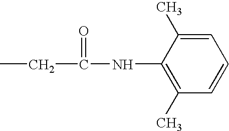 | (±)-trans; mp 185.8° C. |
| 54 | B.5 | 1 | 1 | 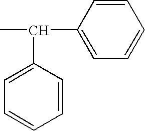 | (±)-cis; mp 260° C. |
| 55 | B.5 | 1 | 1 | 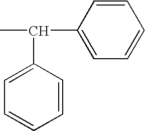 | (±)-trans; mp 75.2° C. |
| 56 | B.5 | 1 | 1 | 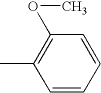 | (±)-trans; mp 80.1° C. |
| 57 | B.5 | 1 | 1 | 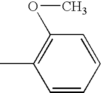 | (±)-cis |
| 58 | B.1 | 1 | 1 | 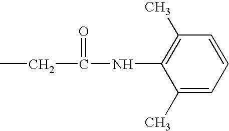 | (±) |
| 59 | B.4 | 1 | 1 | 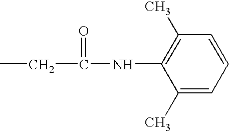 | (±)-cis; mp 106.4° C. |
| 60 | B.6 | 1 | 1 | 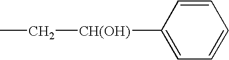 | (±)-cis |
| 61 | B.6 | 1 | 1 | 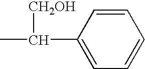 | (±)-cis |
| 62 | B.1 | 1 | 1 | 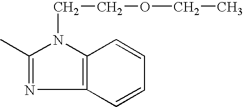 | (±)-cis |
| 63 | B.1 | 1 | 2 | 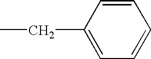 | (±)-cis;fumaric acid (1:2) |
| 64 | B.2 | 1 | 2 | —H | (±)-cis |
| 65 | B.4 | 1 | 2 | 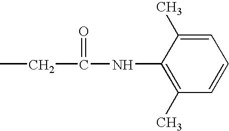 | (±)-cis |
| 66 | B.1 | 1 | 1 | 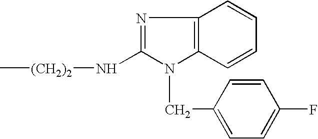 | (±)-cis |
| 67 | B.1 | 1 | 1 | 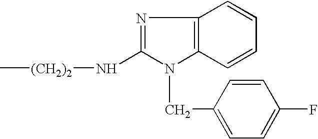 | (±)-trans |
| 68 | B.1 | 1 | 2 | 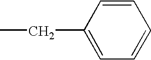 | (±)-trans; fumaric acid(1:2) |
| 69 | B.4 | 1 | 1 | 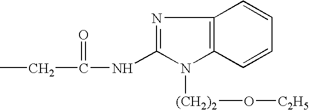 | (±)-trans |
| 70 | B.7 | 1 | 1 | 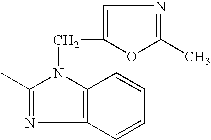 | (±)-cis |
| 71 | B.4 | 1 | 1 | 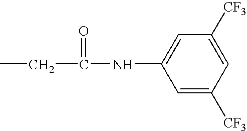 | (±)-cis |
| 72 | B.4 | 1 | 1 | 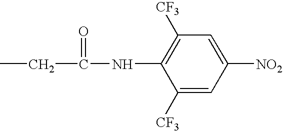 | (±)-cis |
| 73 | B.4 | 1 | 1 | 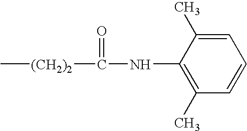 | (±)-cis; fumaric acid (1:2) |
| 74 | B.4 | 1 | 1 | 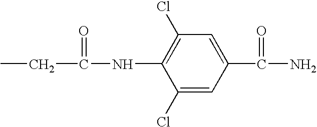 | (±)-cis |
| 75 | B.4 | 1 | 1 | 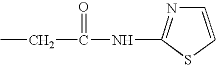 | (±)-cis; fumaric acid (1:1) |
| 76 | B.4 | 1 | 1 | —CH₂—C(═O)—NH—CH(CH₃)₂ | (±)-cis |
| 77 | B.2 | 1 | 2 | —H | (±)-trans |
| 78 | B.4 | 1 | 2 | 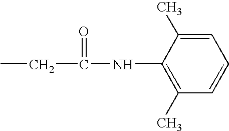 | (±)-trans |
| 79 | B.8 | 1 | 1 | 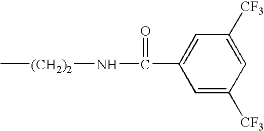 | (±)-cis |
| 80 | B.1 | 1 | 1 | 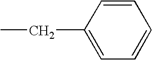 | (+)-(B)-trans |
| 81 | B.2 | 1 | 1 | —H | (+)-(B)-trans |
| 82 | B.1 | 1 | 1 | 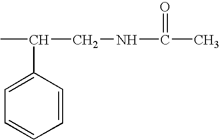 | (±)-cis |
| 83 | B.1 | 1 | 1 | 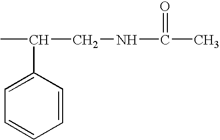 | (±)-trans |
| 84 | B.4 | 1 | 1 | 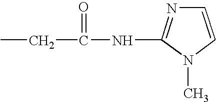 | (±)-cis |
| 85 | B.7 | 1 | 1 | 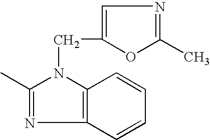 | (±)-trans |
| 86 | B.6 | 1 | 1 | 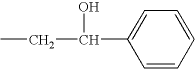 | (±)-trans |
| 87 | B.1 | 1 | 1 | 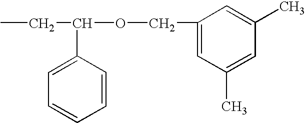 | (±)-trans |
| 88 | B.1 | 1 | 1 | 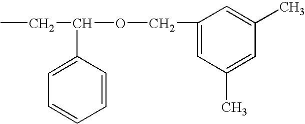 | (±)-cis;fumaric acid (1:2) |
| 89 | B.4 | 1 | 1 | —(CH₂)₂—OH | (±)-trans |
| 90 | B.11 | 1 | 1 | 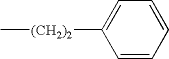 | (±)-trans; fumaric acid(1:2) |
| 91 | B.4 | 1 | 1 | 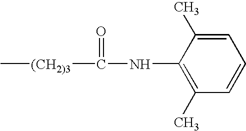 | (±)-trans; fumaric acid(1:2) |
| 92 | B.11 | 1 | 1 | 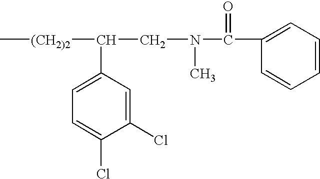 | (±)-trans |
| 93 | B.11 | 1 | 1 | 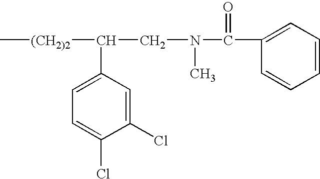 | (±)-cis; fumaric acid (1:2) |
| 94 | B.4 | 1 | 1 | 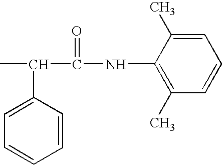 | (±)-trans |
| 95 | B4 | 1 | 1 | 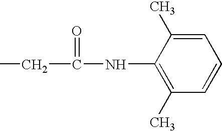 | (B)-trans; (L)-malic acid(1:1) |
| 96 | B.4 | 1 | 1 | 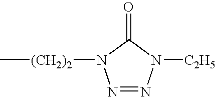 | (±)-trans; fumaric acid(1:2) |
| 97 | B.4 | 1 | 1 | 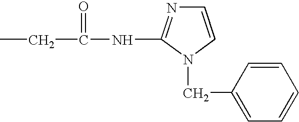 | (±)-trans |
| 98 | B.4 | 1 | 1 | 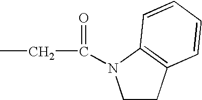 | (±)-cis |
| 99 | B.4 | 1 | 1 | 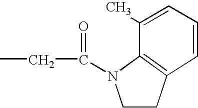 | (±)-cis |
| 100 | B.4 | 1 | 1 | 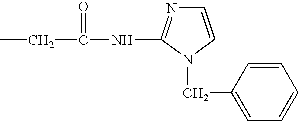 | (±)-cis |
| 101 | B.4 | 1 | 1 | 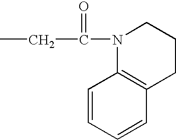 | (±)-cis |
| 102 | B.4 | 1 | 1 | 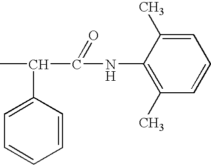 | (±)-cis |
| 103 | B.4 | 1 | 1 | 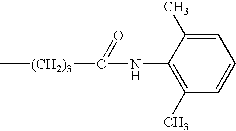 | (±)-cis fumaric acid (1:2) |
| 104 | B.4 | 1 | 1 | 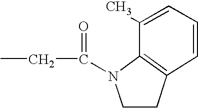 | (±)-trans |
| 105 | B.4 | 1 | 1 | 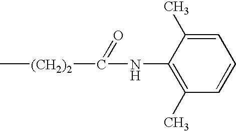 | (±)-trans |
| 106 | B.4 | 1 | 1 | 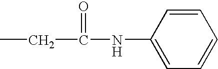 | (±)-trans |
| 107 | B.4 | 1 | 1 | 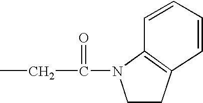 | (±)-trans |
| 108 | B.4 | 1 | 1 | 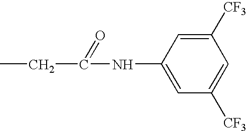 | (±)-trans |
| 109 | B.4 | 1 | 1 | 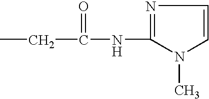 | (±)-trans |
| 110 | B.14 | 1 | 1 | 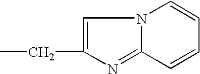 | (±)-trans; fumaric acid(1:2) |
| 111 | B.14 | 1 | 1 | 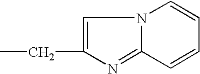 | (±)-cis; fumaric acid (1:2) |
| 112 | B.4 | 1 | 1 | 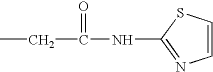 | (±)-trans |
| 113 | B.1 | 1 | 1 | 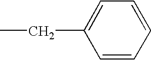 | (±)-trans |
| 114 | B.2 | 1 | 1 | H | (±)-trans; fumaric acid(1:2) |
| 115 | B.8 | 1 | 1 | 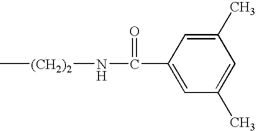 | (±)-trans |
| 116 | B.8 | 1 | 1 | 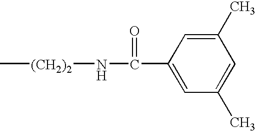 | (±)-cis |
| 117 | B.8 | 1 | 1 | 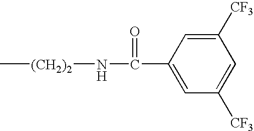 | (±)-trans |
| 118 | B.4 | 1 | 1 | 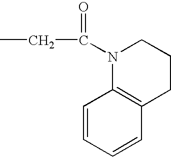 | (±)-trans; fumaric acid(1:2) |
| 119 | B.4 | 1 | 1 | 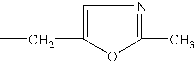 | (±)-trans |
| 120 | B.13 | 1 | 1 | 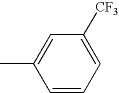 | (±)-trans |
| 121 | B.1 | 1 | 1 | 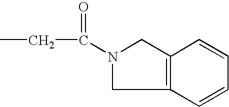 | (±)-cis |
| 122 | B.1 | 1 | 1 | 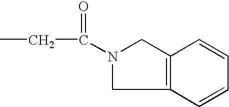 | (±)-trans |
| 123 | B.4 | 1 | 1 | 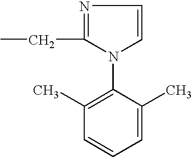 | (±)-cis |
| 124 | B.15 | 1 | 1 | 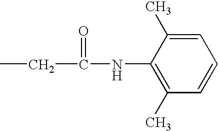 | (B)-trans, benzoate (1:1) |
| 125 | B.15 | 1 | 1 | 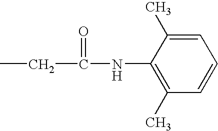 | (B)-trans; maleic acid (1:1) |
| 126 | B.15 | 1 | 1 | 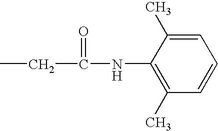 | (B)-trans;hydrochloric acid (1:2)hydrate (1:1) |
| 127 | B.15 | 1 | 1 | 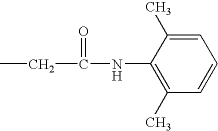 | (B)-trans;succinic acid (1:1) |
| 128 | B.15 | 1 | 1 | 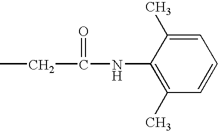 | (B)-trans;fumaric acid (1:1) |
| 129 | B.9 | 1 | 1 | 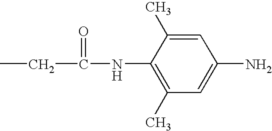 | (±)-cis; fumaric acid (1:2) |
| 130 | B.10 | 1 | 1 | —CH₃ | (±)-cis; fumaric acid (1:2) |
| 131 | B.16 | 0 | 1 | 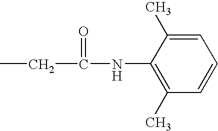 | (±)-cis |

TABLE 3

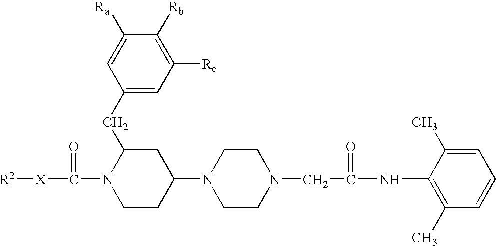

| Co. | | | | | | | |
|---|---|---|---|---|---|---|---|
| No. | Ex. | X | R² | Rₐ | R_{b} | R_{c} | Physical data |
| 132 | B.12 | c.b. | 3,5-di(trifluoro- | F | H | F | (±)-cis |
| | | | methyl)phenyl | | | | |
| 133 | B.12 | c.b. | 3,5-di(trifluoro- | F | H | F | (±)-trans |
| | | | methyl)phenyl | | | | |
| 134 | B.12 | c.b. | 3,5-di(trifluoro- | H | F | F | (±)-cis |
| | | | methyl)phenyl | | | | |
| 135 | B.12 | c.b. | 3,5-di(trifluoro- | H | F | F | (±)-trans |
| | | | methyl)phenyl | | | | |
| 136 | B.4 | c.b. | 3,5-di(trifluoro- | H | CF₃ | H | (±)-(B) fumaric |
| | | | methyl)phenyl | | | | acid (1:1) |
| 137 | B.4 | c.b. | 3,5-di(trifluoro- | H | CF₃ | H | (±)-(A) |
| | | | methyl)phenyl | | | | |
| 138 | B.13 | c.b. | 3,5-di(trifluoro- | H | Cl | Cl | (±)-cis |
| | | | methyl)phenyl | | | | |
| 139 | B.13 | c.b. | 3,5-di(trifluoro- | H | Cl | Cl | (±)-trans |
| | | | methyl)phenyl | | | | |
| 140 | B.19 | c.b. | 3,5-di(trifluoro- | F | H | CF₃ | (±)-cis |
| | | | methyl)phenyl | | | | |
| 141 | B.19 | c.b. | 3,5-di(trifluoro- | F | H | CF₃ | (±)-trans |
| | | | methyl)phenyl | | | | |
| 142 | B.13 | c.b. | 3-isopropoxyphenyl | H | H | H | (±)-cis |
| 143 | B.18 | —NH— | 3,5-di(trifluoro- | H | H | H | (±)-trans |
| | | | methyl)phenyl | | | | |
| 144 | B.13 | c.b. | phenyl | H | H | H | (±)-cis |
| 145 | B.13 | c.b. | 2-naphtyl | H | H | H | (±)-trans |
| 146 | B.13 | c.b. | 2-quinolinyl | H | H | H | (±)-trans |
| 147 | B.13 | c.b. | 2-quinoxalinyl | H | H | H | (±)-trans |
| 148 | B.13 | —O— | benzyl | H | H | H | (±)-trans |
| 149 | B.17 | c.b. | 3-methyl- | H | H | H | (±)-trans |
| | | | benzofuran-2-yl | | | | |
| 150 | B.17 | c.b. | 5-fluoro-indol-2-yl | H | H | H | (±)-trans |
| 151 | B.17 | c.b. | 5-indolyl | H | H | H | (±)-trans |
| 152 | B.17 | c.b. | 5-methyl-pyrazin-2- | H | H | H | (±)-trans |
| | | | yl | | | | |
| 153 | B.13 | c.b. | phenyl | H | H | H | (±)-trans |
| 154 | B.13 | c.b. | 5-methyl-isoxazol-3- | H | H | H | (±)-trans |
| | | | yl | | | | |
| 155 | B.13 | c.b. | 2,4,6- | H | H | H | (±)-cis |
| | | | trimethylphenyl | | | | |
| 156 | B.13 | c.b. | 3,4,5- | H | H | H | (±)-cis |
| | | | trimethoxyphenyl | | | | |
| 157 | B.13 | c.b. | 3-cyanophenyl | H | H | H | (±)-cis |
| 158 | B.13 | c.b. | 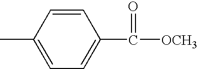 | H | H | H | (±)-cis |
| 159 | B.13 | c.b. | 3,5-difluorophenyl | H | H | H | (±)-cis |
| 160 | B.13 | c.b. | 2,6-dichloro-pyridin- | H | H | H | (±)-cis |
| | | | 4-yl | | | | |
| 161 | B.13 | c.b. | 2-naphtyl | H | H | H | (±)-cis |
| 162 | B.13 | c.b. | 2-quinolinyl | H | H | H | (±)-cis |
| 163 | B.13 | c.b. | 3-isopropoxybenzyl | H | H | H | (±)-cis |
| 164 | B.13 | c.b. | 1-phenylethyl | H | H | H | (±)-cis |
| 165 | B.13 | c.b. | 3-isopropoxyphenyl | H | H | H | (±)-trans |
| 166 | B.13 | c.b. | 3-cyanophenyl | H | H | H | (±)-trans |
| 167 | B.13 | c.b. | 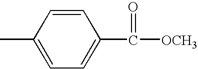 | H | H | H | (±)-trans |
| 168 | B.13 | c.b. | 2,4-dichlorophenyl | H | H | H | (±)-trans |
| 169 | B.13 | c.b. | 2-thienyl | H | H | H | (±)-trans |

c.b. = covalent bond

TABLE 4

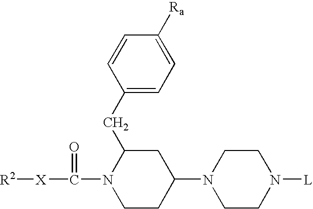

| Co.No. | Ex. | Rₐ | X | R² | L | Physical data |
|---|---|---|---|---|---|---|
| 170 | B.1 | CF₃ | c.b. | 3,5-di(trifluoromethyl)phenyl | H | (±)-(A) |
| 171 | B.1 | CF₃ | c.b. | 3,5-di(trifluoromethyl)phenyl | H | (±)-(B) fumaric |
| | | | | | | acid (1:4) |
| 172 | B.13 | H | c.b. | 3-trifluoromethyl-phenyl | benzyl | (±)-cis |
| 173 | B.13 | H | c.b. | 3,5-difluoro-phenyl | benzyl | (±)-cis |
| 174 | B.13 | H | c.b. | 2-naphtyl | benzyl | (±)-cis |
| 175 | B.13 | H | c.b. | 3-cyano-phenyl | benzyl | (±)-cis |
| 176 | B.13 | H | —O— | benzyl | benzyl | (±)-cis |
| 177 | B.13 | H | c.b. | 2-furanyl | benzyl | (±)-cis |
| 178 | B.13 | H | c.b. | 2-thienyl | benzyl | (±)-cis |
| 179 | B.13 | H | c.b. | phenyl | benzyl | (±)-cis |
| 180 | B.13 | H | c.b. | 3,5-dichloro-phenyl | benzyl | (±)-cis |
| 181 | B.17 | H | c.b. | 3,4-dichlorophenyl | benzyl | (±)-cis |
| 182 | B.13 | H | c.b. | 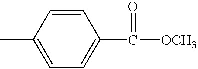 | benzyl | (±)-cis |
| 183 | B.13 | H | c.b. | phenyl | benzyl | (±)-trans |
| 184 | B.13 | H | c.b. | 2,6-dichloro-pyridin-4-yl | benzyl | (±)-trans |
| 185 | B.13 | H | c.b. | 2-furanyl | benzyl | (±)-trans |
| 186 | B.13 | H | c.b. | 2-thienyl | benzyl | (±)-trans |
| 187 | B.13 | H | c.b. | 3-cyano-phenyl | benzyl | (±)-trans |
| 188 | B.13 | H | c.b. | 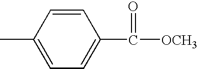 | benzyl | (±)-trans |
| 189 | B.13 | H | c.b. | 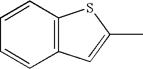 | benzyl | (±)-trans |
| 190 | B.13 | H | c.b. | 5-methyl-isoxazol-3-yl | benzyl | (±)-trans |
| 191 | B.13 | H | c.b. | 2-nitrophenyl | benzyl | (±)-cis |
| 192 | B.16 | H | c.b. | 2-aminophenyl | benzyl | (±)-cis fumaric |
| | | | | | | acid (1:2) |

c.b. = covalent bond

### C. Pharmacological Examples

### C.1: NK₁-Antagonism

The NK₁-antagonistic activity of the compounds in the Tables 1 to 4 has been disclosed in WO 97/16440-A1 which is disclosed herein by reference. Unless otherwise specified, all tests were performed with Compound 95: ((±)-(B)-trans-4-[1-[3,5-bis(trifluoromethyl)benzoyl]-2-(phenylmethyl)-4-piperidinyl]-N-(2,6-dimethylphenyl)-1-piperazine acetamide, (L)-malic acid (1:1)).

### C.2: Inhibition of Morphine-Induced Emesis in Ferrets

In order to test whether a NK₁, antagonist was also able to overcome emesis when co-administered with a centrally acting opioid, ascending doses of Compound 95 were tested against different concentrations of morphine in non food-deprived ferrets after i.p. administration.

Based on preliminary data, doses of 0.4 and 0.8 mg/kg morphine were selected. Increasing the dose from 0.4 to 0.8 mg/kg morphine resulted in more animals with emesis and increased frequencies of periods of emesis, retches and vomiting (Table 5). Doses>0.8 mg/kg morphine caused central side-effects.

### At 0.4 mg/kg morphine, a submaximal dose which did not result in emesis in all animals tested, there was a non-significant tendency with Compound 95 to reduce the frequency of retches, vomiting and periods of emesis.

At 0.8 mg/kg morphine, a more robust drug-induced emesis was present. Doses of 10 and 40 mg/kg i.p. Compound 95 significantly reduced the number of retches. The number (frequency) of vomits diminished significantly in the presence of 10 mg/kg Compound 95 i.p. In terms of periods of emesis, activity was seen at doses>2.5 mg/kg Compound 95.

### These results indicate that NK₁, antagonists can overcome opioid-induced emesis even when co-administered with the opioids.

TABLE 5
Effects of increasing doses of Compound 95 on morphine-induced emesis in ferrets

| | Dose | | | Frequency | | | |
|---|---|---|---|---|---|---|---|
| | Compound 95 | Latency of onset (sec) | | | | Periods of | Duration of |
| Challenge | (mg/kg i.p.) | Retching | Vomiting | Retches | Vomiting | emesis | emesis (sec) |
| Morphine | Vehicle | 357.86 ± (90.99) | 471.67 ± (123.98) | 22.88 ± (6.49) | 2.63 ± (0.92) | 4.38 ± (0.94) | 6.39 ± (0.95) |
| 0.4 mg/kg i.p. | 0.16 | 217.00 ± (50.62) | 290.80 ± (52.25) | 28.00 ± (10.70) | 2.50 ± (1.07) | 4.88 ± (1.59) | 7.37 ± (1.63) |
| n = 8 per group | 0.63 | 247.17 ± (43.87) | 263.6 ± (51.71) | 36.25 ± (13.97) | 3.25 ± (1.39) | 5.38 ± (2.03) | 8.90 ± (1.35) |
| | 2.5 | 313.75 ± (64.34) | 502.25 ± (234.81) | 17.88 ± (8.50) | 2.38 ± (1.18) | 3.13 ± (1.41) | 9.10 ± (2.16) |
| | 10 | 329.60 + (63.10) | 373.25 + (33.99) | 12.63 + (5.95) | 1.00 + (0.50) | 2.63 + (1.19) | 6.78 + (1.21) |
| Morphine | Vehicle | 196.25 ± (26.43) | 218.00 ± (24.03) | 45.25 ± (10.12) | 5.25 ± (1.56) | 9.50 ± (2.17) | 8.41 ± (1.07) |
| 0.8 mg/kg i.p. | 0.63 | 216.38 ± (31.51) | 347.00 ± (30.44)* | 25.50 ± (6.18) | 1.50 ± (0.65) | 4.75 ± (1.26) | 9.05 ± (1.24) |
| n = 8 per group | 2.5 | 251.57 ± (27.52) | 301.20 ± (54.78) | 22.75 ± (5.53) | 1.88 ± (0.79) | 3.50 ± (0.71)* | 6.20 ± (1.50) |
| | 10 | 353.29 ± (94.38) | 399.75 ± (122.43) | 15.25 ± (4.39)* | 0.88 ± (0.35)* | 3.50 ± (0.98)* | 5.20 ± (1.13) |
| | 40 | 275.50 ± (84.10) | 348.25 ± (74.57) | 9.63 ± (5.40)** | 1.13 ± (0.61) | 3.00 ± (1.55)* | 5.10 ± (1.07) |

Given are the mean (SEM) results of 8 ferrets/condition treated simultaneously i.p. with various doses of Compound 95 and morphine.
Differences from vehicle controls were evaluated using the MWU test (two-tailed; *p < 0.05; **p < 0.01)

### C.3: Respiratory Depression

In order to evaluate the effects of combinations of opioids with NK₁ antagonists on respiratory depression, experiments were performed in gerbils, guinea pigs and rats, measuring arterial blood gases, and especially opioid-induced P_{CO}_{₂} increases over time after drug treatment using a blood gas analyzer (ABL Radiometer Copenhagen).

On the day of testing, male gerbils were equipped with a catheter in the arteria femoralis. After full recovery from surgery and habituation to the test conditions, pre-drug baselines were taken. Only animals with P_{CO}_{₂} blood levels<30 mmHg were included for drug testing. Drugs were injected s.c. and samples were taken at repeated intervals after treatment.
Opioids resulted in an increase in P_{CO}_{₂} values over time. With Compound 95, up to 10 mg/kg s.c., no effects were observed. Adding 10 mg/kg s.c. Compound 95 to the highest doses of fentanyl (0.63 mg/kg s.c.) and morphine (10 mg/kg s.c.), resulted in significant decreases in the opioid-induced P_{CO}_{₂} levels

These data indicate that Compound 95 can reduce part of the respiratory depression induced by opioids. Furthermore, a role of SP in the respiratory system is suggested in the literature. If the effects of Compound 95 reflect here an antagonism of the opioid-induced excitation, this can be beneficial because opioid-induced excitation is sometimes seen at overdosing.

### In both guinea pigs and rats, opioids do not result in excitation and therefore the effects of Compound 95 on the opioid-induced P_{CO}_{₂} levels should be more directly related to effects in the respiratory system.

Guinea pigs were chronically catheterized with an intra-arterial line into the arteria carotis. In order to keep the lines patent, the animals were connected to chronic infusion pumps in their home cages and minimal amounts of heparine in saline were administered daily. After full recovery of the animals and habituation to the chronic housing conditions, the animals were disconnected from the pumps and after s.c. drug treatment, samples were taken in their home cages at repeated time intervals.
Both fentanyl and morphine resulted in an increase in P_{CO}_{₂}. With fentanyl, higher increases in P_{CO}_{₂} were generally measured. Compound 95, tested at doses ranging from 0.16 to 10 mg/kg s.c., did not affect P_{CO}_{₂} values. Adding Compound 95 to both fentanyl and morphine resulted in reductions in the opioid-induced P_{CO}_{₂} levels . The effects were more pronounced with fentanyl, probably due to the high doses tested.
Rats were acutely equipped with a catheter in the arteria femoralis. After full recovery from surgery and habituation to the test conditions, predrug baseline values were taken. Only animals with P_{CO}_{₂} blood levels<35 mm Hg were included. Drugs were injected s.c. and samples were taken at repeated intervals after treatment.
As in the other species, the opioids fentanyl and morphine both resulted in increased levels of P_{CO}_{₂} at the highest doses tested. Compound 95 at 40 mg/kg s.c. did not affect the P_{CO}_{₂} levels. Adding 40 mg/kg s.c. Compound 95 to the opioids resulted in some attenuation of the opioid-induced increases in P_{CO}_{₂}, especially at the highest doses tested. Further analysis indicated that Compound 95 especially reduced the number of animals reaching high (>50 mm Hg) levels of P_{CO}_{₂}.
Thus, the data in rats confirm the observations in guinea pigs and gerbils that the NK₁ antagonist Compound 95 can reduce the opioid-induced increases in P_{CO}_{₂}, pointing to some functional role of NK₁ antagonists on the opioid-induced respiratory depression. ps C.4: Constipation: The Castor Oil Test

In order to evaluate the constipatory effects of opioids in combination with NK₁, antagonists, a castor oil test was performed in gerbils and rats. In both species, and one hour after s.c. pretreatment with the test compounds, animals were challenged with 0.25 (gerbils) or 1 ml (rats) ricinus oil, and the number of animals showing diarrhea were measured every hour up to 8 hours after the challenge. Because, in control animals (treated with vehicle), >95% revealed diarrhea within 4 (rats) and 8 h (gerbils), respectively, these time points were used to calculate ED₅₀s for opioid-induced constipation (opioid-induced inhibition of diarrhea).

All opioids tested (morphine, fentanyl, codeine and oxycodone in gerbils and morphine and fentanyl in rats) resulted in a dose-dependent inhibition of the ricinus oil-induced diarrhea (Table 6). Compound 95, at doses up to 40 mg/kg s.c., had minimal effects on constipation in both rats and gerbils.

Adding Compound 95 to the opioids did not alter the ED₅₀s of the opioids, neither in gerbils nor in rats (Table 6).

TABLE 6
Effects of opioids and Compound 95 in the castor oil test in gerbils and
rats.

| | gerbils | | | rats | |
|---|---|---|---|---|---|
| | | Compound 95 | Compound 95 | | Compound 95 |
| Compound | vehicle | 0.16 mg/kg s.c. | 2.5 mg/kg s.c. | vehicle | 40 mg/kg s.c. |
| Fentanyl | 0.080 | 0.14 | 0.020 | 0.11 | 0.11 |
| | (0.036-0.18) | (0.062-0.31) | (0.009-0.044) | (0.06-0.19) | (0.05-0.24) |
| Morphine | 0.72 | 1.66 | 0.32 | 2.19 | 0.95 |
| | (0.29-1.89) | (0.50-5.48) | (0.096-1.05) | (1.19-4.01) | (0.52-1.75) |
| Oxycodone | 5.02 | 11.51 | 5.02 | — | — |
| | (1.92-13.1) | (6.28-21.11) | (2.84-11.27) | | |
| Codeine | 1.67 | 1.67 | 2.20 | — | — |
| | (0.64-4.35) | (0.74-3.74) | (0.38-4.93) | | |

Given are the ED₅₀s (95% CL) values in mg/kg of the opioids to postpone the ricinus oil-induced diarrhea for 8 (gerbils) and 4 h. (rats) respectively. For each test condition n = 5 were used. No differences between the ED₅₀s of the opioid and the respective combinations with Compound 95 were observed.

### C.5: Gastrointestinal Transit: The Charcoal Test

In order to evaluate the effects of adding an NK₁, antagonist to the effects of opioids on gastrointestinal transit, a charcoal test was performed in male gerbils and rats. For testing, the animals were pretreated s.c. with the test compound, 1 h prior to the oral administration of 1 ml (gerbil) or 2 ml (rat) charcoal (10% in 5% Arabic gum) per 100 g body weight. After 20 min (gerbil) and 30 min (rat), the animals were sacrificed, the charcoal propulsion was measured throughout the small intestine and the peristalsis ratio was calculated. This is the ratio between the distance attained by the charcoal in the small intestines (as measured from the end of the stomach (pylorus)) and the total length of the small intestines (up to the beginning of the caecum). Because, in control rats (n=30) and gerbils (n=155), a peristalsis ratio<60% was never observed, this level was used as an all or nothing criterion to calculate GI tract inhibition.

In gerbils, all opioids resulted in a dose-dependent inhibition of the charcoal propulsion while Compound 95 was inactive. Adding Compound 95 to the opioids did not affect the outcome of the opioids (Table 7).

In rats, comparable results were obtained with morphine and Compound 95. With fentanyl, tested up to 0.16 mg/kg s.c., no inhibition of the charcoal transit was observed, also when combined with 40 mg/kg s.c. Compound 95 (Table 7).

TABLE 7
Effects of opioids and Compound 95 in the charcoal test in gerbils and
rats.

| | gerbils | | | rats | |
|---|---|---|---|---|---|
| | | Compound 95 | Compound 95 | | Compound 95 |
| | vehicle | 0.16 mg/kg s.c. | 2.5 mg/kg s c | vehicle | 40 mg/kg s.c. |
| Fentanyl | 0.032 | 0.043 | 0.057 | >0.16 | >0.16 |
| | (0.022-0.049) | (0.032-0.058) | (0.042-0.077) | | |
| Morphine | 0.043 | 0.037 | 0.057 | 1.66 | 3.81 |
| | (0.032-0.058) | (0.028-0.051) | (0.042-0.077) | (0.74-3.72) | (2.08-6.98) |
| Oxycodone | 1.26 | 2.19 | 1.66 | — | — |
| | (0.82-1.93) | (0.97-4.90) | (0.63-4.32) | | |
| Codeine | 0.9 | 1.26 | 0.41 | — | — |
| | (0.42-2.14) | (0.48-3.28) | (0.14-0.23) | | |

Given are the ED₅₀s (95% CL) in mg/kg of the opioids for an inhibition of charcoal propulsion (<60%). No differences between the ED₅₀s of the opioids and the respective combinations were observed.

### Taken together as indices for GI activity, the castor oil and the charcoal test results in both rats and gerbils confirm that opioids have constipatory effects, which were not potentiated by Compound 95.

### C.6: Tolerance in a Chronic Neuropathic Pain Model

In order to evaluate the effects of adding a NK₁ antagonist on the opioid-induced tolerance over time in a chronic pain model, experiments were performed in gerbils with a chronic constrictive injury (CCI) using the acetone spray test. In CCI gerbils, acetone is demonstrated to produce an increased pain reactivity (cold/chemical hyperalgesia). This pain reactivity can be acutely antagonized by opioids, but not by NK₁, antagonists.

To test whether Compound 95 could prevent the loss of efficacy of morphine over time in this model, CCI gerbils were tested over an 11 days period receiving twice daily i.p. injections of either vehicle, 2.5 mg/kg morphine, 2.5 mg/kg Compound 95 or the combination of 2.5 mg/kg morphine with 2.5 mg/kg Compound 95.

As was seen, morphine acutely reduced the acetone-induced lifting behaviour but reductions of activity became apparent from the second day onwards. At day 7, no differences with the controls were present anymore. With Compound 95, limited activity was present during some days of the experiment. The combination of Compound 95 with morphine resulted in an initial activity at the first day similar to morphine alone, and as opposed to morphine, the activity remained present over the entire testing period.

### These data indicate that, under certain testing conditions, an NK₁, antagonist can overcome the loss of efficacy of an opioid over time, pointing to the prevention of the development of tolerance to the functional effects of these drugs.

### C.7: Discriminative Stimulus Properties of Opioids

In order to evaluate whether a NK₁, antagonist could interfere with the abuse potential, and especially the central narcotic properties of opioids, the effects of Compound 95 on the discriminative stimulus properties of fentanyl were studied in rats trained to discriminate 0.04 mg/kg s.c. fentanyl from saline in a two lever food reinforced test procedure.

The ED₅₀ (95% CL) values of fentanyl for stimulus generalization in fentanyl trained rats injected s.c. 30 and 60 min prior to testing were 0.018 (0.012-0.028) and 0.021 (0.016-0.029) mg/kg, respectively.

### Compound 95 at doses ranging from 0.63 to 40 mg/kg s.c. did not show any generalization to nor any antagonism of the fentanyl cue.

Pretreatment with 10 or 40 mg/kg Compound 95 did not affect the stimulus generalization of fentanyl in the fentanyl trained rats. No shifts in the dose response curves of fentanyl were observed, neither was there a change in the ED₅₀ values for stimulus generalization (Table 8).

TABLE 8
Effects of Compound 95 on the cueing properties of fentanyl in rats.
Given in mg/kg are the ED₅₀s (95% CL) of fentanyl for stimulus
generalization after pretreatment with various doses of Compound
95 in rats trained to discriminate 0.04 mg/kg fentanyl from saline
in a two lever food reinforced drug discrimination test procedure

| Pre-treatment | |
|---|---|
| (s.c., 60 min prior to test) | ED₅₀ (mg/kg) |
| Vehicle | 0.0193 (0.014-0.027) |
| 10 mg/kg Compound 95 | 0.016 (0.011-0.021) |
| 40 mg/kg Compound 95 | 0.013 (0.0085-0.019) |

These results clearly indicate that, in rats, Compound 95 does not interfere with the discriminative stimulus properties of the opioid fentanyl.

1. A pharmaceutical composition comprising a pharmaceutically acceptable carrier and, as active ingredients, an opioid analgesic and a therapeutically effective amount of a compound according to Formula (I) 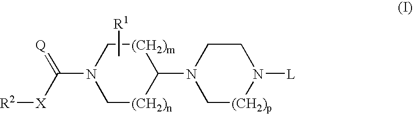
the pharmaceutically acceptable acid or base addition salts thereof, the stereochemically isomeric forms thereof, the N-oxide form thereof and the prodrugs thereof, wherein
n is 0, 1 or 2;
m is 1 or 2, provided that if m is 2, then n is 1;
p is 1 or 2;
═Q is ═O or ═NR₃;
X is a covalent bond or a bivalent radical of formula —O—, —S—, —NR³—;
R¹ is Ar¹, Ar¹C₁₋₆alkyl or di(Ar¹)C₁₋₆alkyl, wherein each C₁₋₆alkyl group is optionally substituted with hydroxy, C₁₋₄alkyloxy, oxo or a ketalized oxo substituent of formula —O—CH₂—CH₂—O— or —O—CH₂—CH₂—CH₂—O—;
R² is Ar², Ar²C₁₋₆alkyl, Het¹ or Het¹C₁₋₆alkyl;
R³ is hydrogen or C₁₋₆-alkyl;
L is hydrogen; Ar³; C₁₋₆alkyl; C₁₋₆alkyl substituted with 1 or 2 substituents selected from hydroxy, C₁₋₆alkyloxy, Ar³, Ar³C₁₋₆alkyloxy and Het²; C₃₋₆alkenyl; Ar³C₃₋₆alkenyl; di(Ar³)C₃₋₆alkenyl or a radical of formula 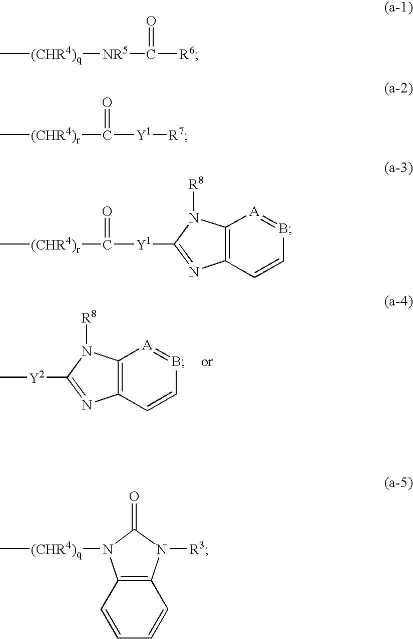
wherein
each q independently is 2, 3 or 4;
each r is0, 1, 2, 3 or 4;
each Y¹ independently is a covalent bond, —O— or NR³;
Y² is a covalent bond, C₁₋₄alkanediyl or -C₁₋₄alkylNR³—;
each -A=B— independently is a bivalent radical of formula —CH═CH—, —N═CH— or —CH═N—;
each R⁴ independently is hydrogen, C₁₋₆alkyl, Ar² or Ar²C₁₋₆alkyl;
R⁵ is hydrogen, C₁₋₆alkyl or Ar³;
R⁶ is C₁₋₆alkyl, Ar³, Ar³C₁₋₆alkyl, di(Ar³)C₁₋₆alkyl, Ar³C₃₋₇cycloalkyl, or indolyl;
R⁷is Ar³C₁₋₆alkyl; di(Ar³)C₁₋₆alkyl; C₁₋₆alkyl; C₃₋₇cycloalkyl; C₃₋₇cycloalkyl substituted with Ar³; oxazolyl; oxazolyl substituted with halo or C₁₋₆alkyl; thiazolyl; thiazolyl substituted with halo or C₁₋₆alkyl; imidazolyl; imidazolyl substituted with Ar³, C₁₋₆alkyl,
Ar³C₁₋₆alkyl or halo; indolinyl; indolinyl substituted with C₁₋₄alkyl; 2,3,4-trihydroquinolinyl; pyrrolidinyl or furanyl;
each R⁸ independently is hydrogen, C₁₋₆alkyl, C₃₋₇cycloalkyl or a radical of formula of formula
-Alk-R¹¹ (b-1),or
-Alk-Z-R¹² (b-2);
wherein
Alk is C₁₋₆alkanediyl;
Z is a bivalent radical of formula —O—, —S— or —NR₃—;
R¹¹ is phenyl; phenyl substituted with 1 or 2 substituents selected from halo, C₁₋₆ alkyl or C₁₋₆alkyloxy; furanyl; furanyl substituted with 1 or 2 substituents selected from C₁₋₆alkyl or hydroxyC₁₋₆alkyl; thienyl; thienyl substituted with 1 or 2 substituents selected from halo or C₁₋₆alkyl; oxazolyl; oxazolyl substituted with 1 or 2 C₁₋₆alkyl substituents; thiazolyl; thiazolyl substituted with 1 or 2 C₁₋₆alkyl substituents; pyridinyl or pyridinyl substituted with 1 or 2 C₁₋₆alkyl substituents;
R¹² is C₁₋₆alkyl or C₁₋₆alkyl substituted with hydroxy, carboxyl or C₁₋₆alklyoxycarbonyl;
Ar¹ is phenyl; phenyl substituted with 1, 2 or 3 substituents each independently selected from the group consisting of halo, C₁₋₄alkyl, haloC₁₋₄alkyl, cyano, aminocarbonyl, C₁₋₄alkyloxy and haloC₁₋₄alkyloxy;
Ar² is naphtalenyl; phenyl; phenyl substituted with 1, 2 or 3 substituents each independently selected from the group consisting of hydroxy, halo, cyano, nitro, amino, mono- or di(C₁₋₄alkyl)amino, C₁₋₄alkyl, haloC₁₋₄alkyl, C₁₋₄alkyloxy, haloC₁₋₄alkyloxy, carboxyl, C₁₋₄alkyloxycarbonyl, aminocarbonyl and mono- and di(C₁₋₄alkyl)aminocarbonyl;
Ar³ is phenyl or phenyl substituted with 1, 2 or 3 substituents selected from the group, consisting of halo, hydroxy, amino, nitro, aminocarbonyl, C₁₋₆alkyl, haloC₁₋₆alkyl and C₁₋₆alkyoxy;
Het¹ is a monocyclic heterocycle selected from pyrrolyl, pyrazolyl, imidazolyl, furanyl, thienyl, oxazolyl, isoxazolyl, thiazolyl, isothiazolyl, pyridinyl, pyrimidinyl, pyrazinyl and pyridazinyl; or a bicyclic heterocycle selected from the group consisting of quinolinyl, quinoxalinyl; indolyl, benzimidazolyl, benzoxazolyl, benzisoxazolyl, benzothiazolyl, benzisothiazolyl, benzofuranyl and benzothienyl; each monocyclic and bicyclic heterocycle may optionally be substituted on a carbon atom by 1 or 2 substituents selected from the group consisting of halo, C₁₋₄alkyl or mono-, di- and tri(halo)methyl, and
Het² is a heterocycle selected from the group consisting of 1,4dihydro-5-oxo-tetrazol-1-yl, imidazo[1,2-a]pyridinyl, oxazolyl and imidazolyl; each of said heterocycles may be substituted with 1 or where possible 2 substituents selected from the group consisting of C₁₋₄alkyl and Ar³.

2. A pharmaceutical composition according to claim 1 wherein L is hydrogen; C₁₋₆alkyl; C₁₋₆alkyl substituted with hydroxy; C₃₋₆alkenyl; Ar³; Ar³C₁₋₆alkyl; di(Ar³)C₁₋₆alkyl; Ar³C₃₋₆alkenyl; di(Ar³)C₁₋₆alkenyl; or a radical of formula (a-1), (a-2), (a-4) or (a-5) wherein:
R⁷ is Ar³; Ar³C₁₋₆alkyl; di(Ar³)C₁₋₆alkyl; C₁₋₆alkyl; C₃₋₇cycloalkyl; C₃₋₇cycloalkyl substituted with Ar³; oxazolyl; oxazolyl substituted with halo or C₁₋₆alkyl; thiazolyl; thiazolyl substituted with halo or C₁₋₆alkyl; imidazolyl; imidazolyl substituted with Ar³, C₁₋₆alkyl, Ar³C₁₋₆alkyl or halo; pyrrolidinyl or furanyl;
Ar³ is is phenyl or phenyl substituted with 1, 2 or 3 substituents selected from halo, hydroxy, amino, aminocarbonyl, C₁₋₆alkyl, haloC₁₋₆alkyl or C₁₋₆alkyloxy;
Het¹ is a monocyclic heterocycle selected from the group consisting of pyrrolyl, pyrazolyl, imidazolyl, furanyl, thienyl, oxazolyl, isoxazolyl, thiazolyl, isothiazolyl, pyridinyl, primidinyl, pyrazinyl and pyridazinyl; or a bicyclic heterocycle selected from the group consisting of quinolinyl, benzimidazolyl, benzoxazolyl, benzisoxazolyl, benzothiazolyl, benzisothiazolyl, benzofuranyl and benzothienyl; each monocyclic and bicyclic heterocycle may optionally be substituted on a carbon atom by 1 or 2 substituents selected from the group consisting of halo, C₁₋₄alkyl or mono-, di- and tri(halo)methyl.

3. A pharmaceutical composition according to claim 1 wherein, R¹ is Ar¹ methyl and attached to the 2-position or R¹ is Ar¹ and attached to the 3-position.

4. A pharmaceutical composition according to claim 1 wherein, R²-X—C(═Q)— moiety is 3,5-di-(trifluoromethyl) phenylcarbonyl.

5. A pharmaceutical composition according to claim 1 wherein,
R¹ is Ar¹C₁₋₆alkyl, R² is phenyl substituted with 2 substituents selected from the group consisting of methyl and trifluoromethyl, X is a covalent bond and ═Q is ═O.

6. A pharmaceutical composition according to claim 1 wherein, n and m are 1 and p is 1 or 2.

7. A pharmaceutical composition according to claim 1 wherein,
R¹ is phenylmethyl; R² is phenyl substituted with 2 substituents selected from the group consisting of methyl and trifluoromethyl; n, m and p are 1; X is a covalent bond; and ═Q is ═O.

8. A pharmaceutical composition according to claim 1 wherein,
L is a radical of formula (a-2) wherein R⁴ is hydrogen or phenyl; r is 0 or 1; Y¹ is a covalent bond, —O— or —NH—; R⁷ is pyrrolidinyl; furanyl; 1-phenylcyclohexanyl; diphenylmethyl, or phenyl substituted with 1, 2 or 3 substituents each independently selected from the group consisting of methyl, methoxy and chloro

9. A pharmaceutical composition according to claim 1 wherein, the pharmaceutical composition comprises a compound selected from the group consisting of:
4-[1-[3,5-bis(trifuoromethyl)benzoyl]-2-(phenylmethyl)-4-piperidinyl]-N-(2,6-dimethylphenyl)-1-piperazine acetamide;
4[1-[3,5-bis(trifluoromethyl)benzoyl]-2-(phenylmethyl)-4-piperidinyl]-N-(1-phenylcyclohexyl)-1-piperazine acetamide;
1-[3,5-bis(trifluoromethyl)benzoyl]-2-(phenylmethyl)-4-[4-[□-(1-pyrrolidinylcarbonyl)benzyl]-1-piperazinyl]piperidine;
1-[3,5-bis(trifluoromethyl)benzoyl]-4-[4-[1-[(2-methyl-5-oxazolyl)methyl]-1H-benzimidazol-2-yl]-1-piperazinyl]-2-(phenylmethyl)piperidine;
4-[1-[3,5-bis(trifluoromethyl)benzoyl]-2-[(4-trifluoromethylphenyl)methyl]-4-piperidinyl]-N-(2,6-dimethylphenyl)-1-piperazine acetamide; and
4-[1-[3,5-bis(trifluoromethyl)benzoyl]-2-[(3,4dichlorophenyl)methyl]-4-piperidinyl]-N-2,6-dimethylphenyl-1-piperazine acetamide.

10. A pharmaceutical composition according to claim 1 wherein, the pharmaceutical composition comprises a compound selected from the group consisting of;
(+)-(B)-trans-4-[1-[3,5-bis(trifluoromethyl)benzoyl]-2(phenylmethyl)-4-piperidinyl]-N-(2,6-dimethylphenyl)-1-piperazine acetamide;
(−)-(B)-cis4[1-[3,5-bis(trifluoromethyl)benzoyl]-2-(phenylmethyl)-4piperidinyl]-N-(2,6-dimethylphenyl)-1-piperazine acetamide; and
(+)-(B)trans-4[1-[3,5-bis(trifluoromethyl)benzoyl]-2-(phenylmethyl)-4-piperidinyl]-N-(2,6-dimethylphenyl)-1-piperazine acetamide (L)-malic acid (1:1).

11. A pharmaceutical composition according to claim 1 wherein, the pharmaceutical composition is formulated for simultaneous, separate or sequential use.

12. A pharmaceutical composition according to claim 1 wherein, the opioid analgesic is one or more compounds selected from the group consisting of alfentanil, buprenorphine, butorphanol, carfentanyl, codeine, diacetylmorphine, dihydrocodeine, fentanyl, hydrocodone, hydromorphone, levorphanol, lofentanyl, meperidine, methadone, morphine, nalbuphine, oxycodone, oxymorphone, pentazocine, propoxyphene, remifentanyl and sufentanyl; and derivatives and pharmaceutical acceptable salts thereof.

13. A pharmaceutical composition according to claim 12 wherein the opioid analgesic is one or more compounds selected from the group consisting of oxycodone, codeine, morphine, fentanyl, buprenorphine, hydrocodone, hydromorphone and pharmaceutical acceptable salts and derivatives thereof.

14. A pharmaceutical composition according to claim 1 wherein, the pharmaceutical composition is in a form suitable to be orally administered.

15. The use of a pharmaceutical composition according to claim 1, for the prevention and/or treatment of pain and/or nociception.

16. The use of a pharmaceutical composition according to claim 1, for the prevention and/or treatment of acute and chronic pain, more in particular in inflammatory, post-operative, emergency room (ER), breakthrough, neuropathic and cancer pain treatments.

17. The use of a pharmaceutical composition according to claim 1, for the prevention and/or treatment of emesis in opioid-based treatments of pain.

18. The use of a pharmaceutical composition according to claim 17 for the prevention and/or treatment of nausea and vomiting in opioid-based treatments of pain.

19. The use of an NK₁-receptor antagonist, in particular an NK₁-receptor antagonist according to Formula (I), the pharmaceutically acceptable acid or base addition salts thereof, the stereochemically isomeric forms thereof, the N-oxide form thereof and prodrugs thereof, for the prevention and/or treatment of respiratory depression in opioid-based treatments of pain.

20. The use of an NK₁-receptor antagonist, in particular an NK₁-receptor antagonist according to Formula (I), the pharmaceutically acceptable acid or base addition salts thereof, the stereochemically isomeric forms thereof, the N-oxide form thereof and prodrugs thereof, for reducing and/or overcoming the tolerance observed with opioids in opioid-based treatments of pain.
